(19) United States
(12) Patent Application Publication
(10) Pub. No.: US 2011/0020843 A1
(43) Pub. Date: Jan. 27, 2011

(54) LABEL-FREE METHODS RELATED TO PHOSPHODIESTERASES

(76) Inventors: Ye Fang, Painted Post, NY (US); Elizabeth Tran, Painted Post, NY (US); Florence Verrier, Corning, NY (US)

Correspondence Address: CORNING INCORPORATED, SP-TI-3-1, CORNING, NY, 14831, US

(21) Appl. No.: 12/838,033

(22) Filed: Jul. 16, 2010

Related U.S. Application Data
(60) Provisional application No. 61/227,611, filed on Jul. 22, 2009.

(51) Int. Cl. G01N 33/53; C12N 5/071

(52) U.S. Cl. 435/7.21; 435/366

(57) ABSTRACT

196. Disclosed are methods of incubating cells on biosensors, and methods using the disclosed incubation techniques to identify PDE4 modulators.

### I. CLAIMING BENEFIT OF PRIOR FILED U.S. APPLICATION

1. This application claims the benefit of U.S. Provisional Application Ser. No. 61/227,611, filed on Jul. 22, 2009. The content of this document and the entire disclosure of publications, patents, and patent documents mentioned herein are incorporated by reference.

### II. BACKGROUND

2. Cyclic nucleotide phosphodiesterases (PDEs) hydrolyze 3,′5′-cyclic nucleotides, including cAMP (cyclic adenosine monophosphate) and cGMP (cyclic guanosine monophosphate), to their corresponding 5′-nucleotide monophosphates AMP and GMP. Both cAMP and cGMP are important second messengers coupling to the G-protein-coupled receptors (GPCRs) and mediate the responses of a variety of hormones and neurotransmitters. PDEs are responsible for terminating cellular responses to hormones and neurotransmitters, which is critical for maintaining proper intracellular signaling events. Inhibitors of PDEs are highly sought. Disclosed are label free methods for identifying molecules which interact with and can modulate PDEs.

### III. SUMMARY

3. The methods described herein are directed towards using label-free biosensor cellular assays for directly and indirectly detecting PDE activity.

### IV. BRIEF DESCRIPTION OF FIGURES

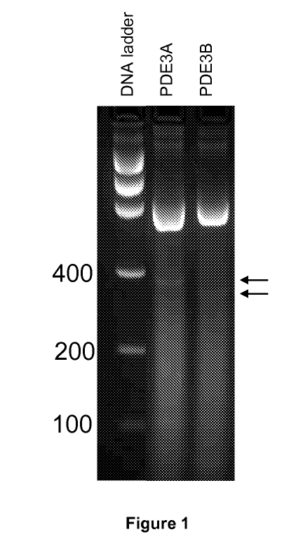

4. FIG. 1 shows that human skin cancerous cell line A431 only expresses low level of PDE3A, and PDE3B, as shown by gel electrophoresis analysis of PCR products of A431 mRNA samples.

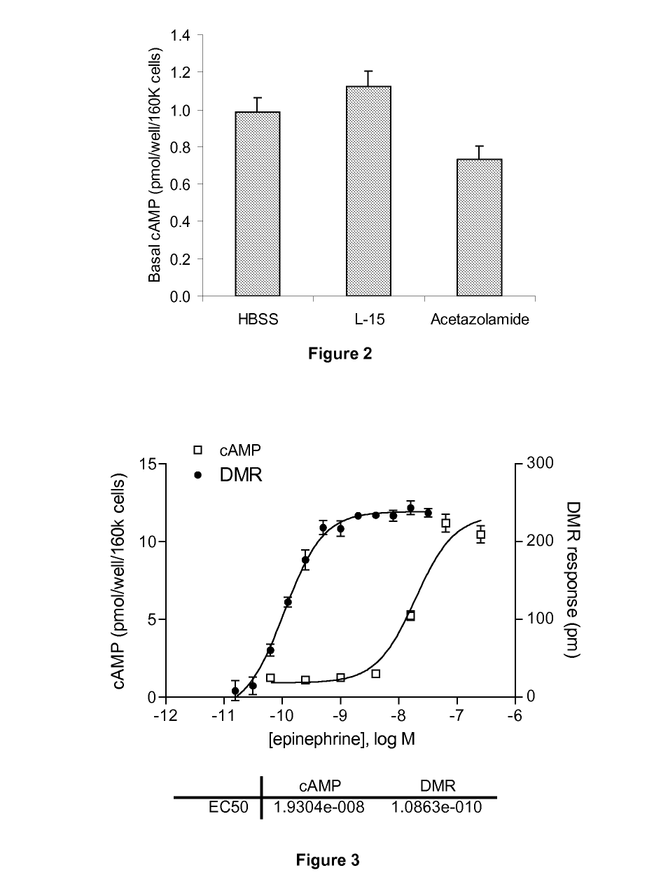

5. FIG. 2 shows the distinct basal cAMP levels of A431 cells under three synchronized conditions: 2 hr incubation in a low CO₂ environment of starved A431 cells maintained using HBSS buffer (HBSS), Leibovitz's L-15 medium CO₂-independent medium (L-15), or HBSS buffer containing 1 micromolar acetazolamide (Acetazolamide).

6. FIG. 3 shows the differential potencies of epinephrine acting on endogenous β2AR in A431 obtained using whole cell lysate cAMP measurement (cAMP) and label-free biosensor cellular assays (DMR response).

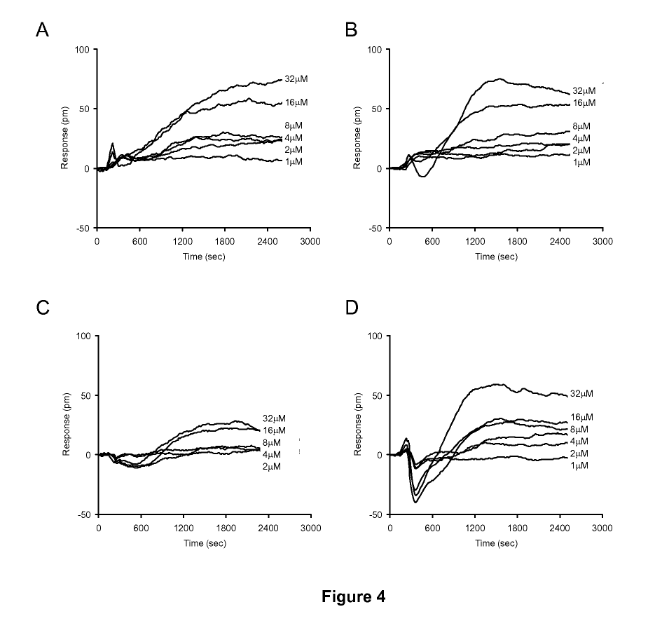

7. FIG. 4 shows the IBMX-induced optical biosensor responses of starved A431 cells under four different synchronization conditions: (A) 2 hr incubation in HBSS buffer, (B) 2 hr incubation in HBSS buffer containing 1 micromolar acetamolamide, (C) 2 hr incubation in the CO₂ independent medium Leibovitz's L-15, and (D) 2 hr incubation in the CO₂ independent medium Leibovitz's L-15 containing 1 micromolar acetamolamide. All incubations were under low (˜1%) CO₂ environment.

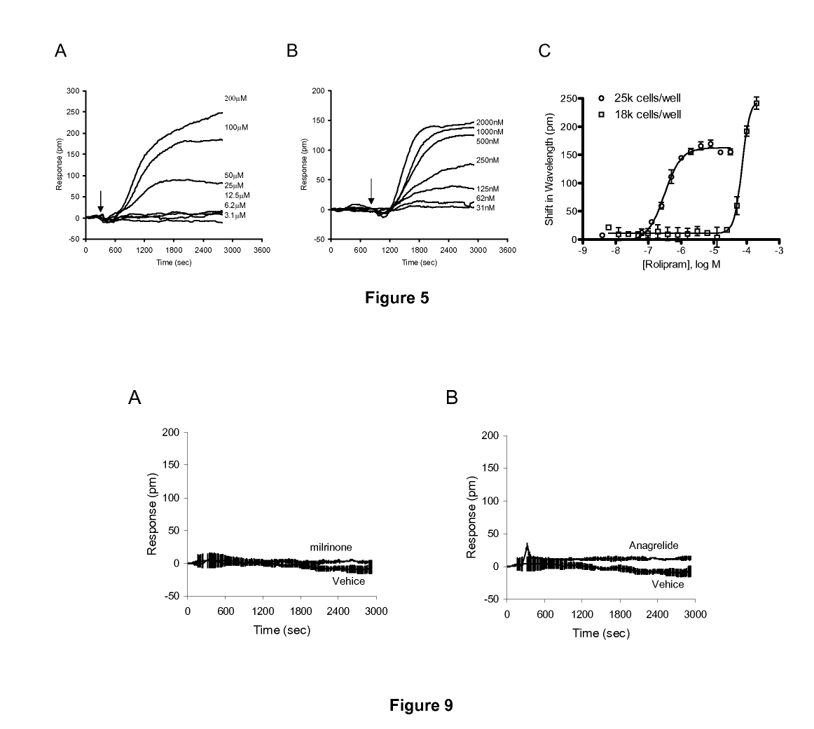

8. FIG. 5 shows the potency of the PDE4 inhibitor R-rolipram depends on the cell synchronization conditions. (A) The dose dependent response of starved A431 cells, wherein the cells were obtained by seeding 18k cells per well in a 384 well biosensor microplate, following by 1 day culture in 10% serum medium and 20 hr starvation in a serum free medium. (B) The dose dependent response of starved A431 cells, wherein the cells were obtained by seeding 25k cells per well in a 384 well biosensor microplate, following by 1 day culture in 10% serum medium and 20 hr starvation in a serum free medium. Before assays, all cells were washed and maintained in the HBSS buffer for 2 hr in a low CO₂ environment. (C) The amplitude, as measured as shift in resonant wavelength in picometer 50 min after stimulation, of the R-rolipram-induced responses as a function of R-rolipram concentrations.

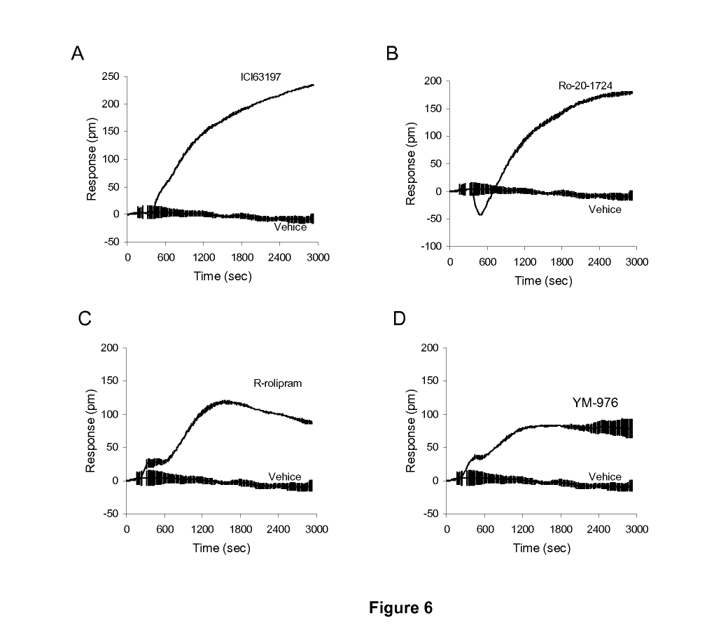

9. FIG. 6 shows examples of the PDE4 specific inhibitors-induced DMR signals of synchronized A431 cells: (A) ICI63197, (B) Ro-20-1724, (C) R-rolipram, and (D) YM-976, in comparison with the DMR signals when the cells were treated with the vehicle only (i.e., the HBSS buffer). The concentrations of all inhibitors were at 12.5 micromolar. The A431 cells were synchronized using the standard protocol: the cells were obtained by seeding 22k cells per well in a 384 well biosensor microplate, following by 1 day culture in 10% serum medium and 20 hr starvation in a serum free medium. Before assays, all cells were washed and maintained in the HBSS buffer for 2 hr in a low CO₂ environment.

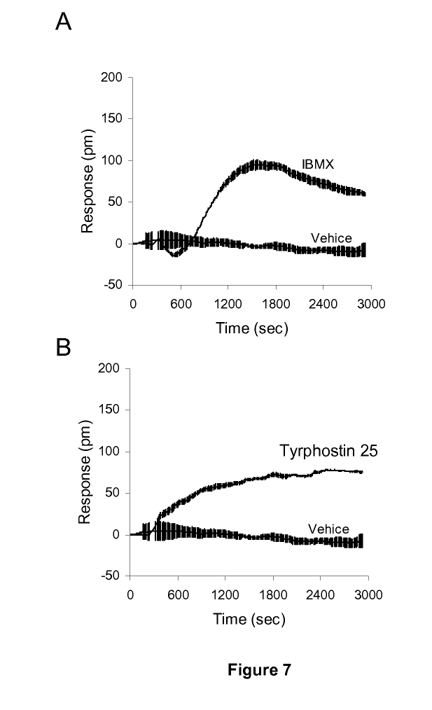

10. FIG. 7 shows examples of the non-selective PDE inhibitors-induced DMR signals of synchronized A431 cells: (A) IBMX, (B) Tyrphostin 25, in comparison with the DMR signals when the cells were treated with the vehicle only (i.e., the HBSS buffer). The concentrations of all inhibitors were at 12.5 micromolar. The A431 cells were synchronized using the standard protocol, same as indicated in FIG. 6.

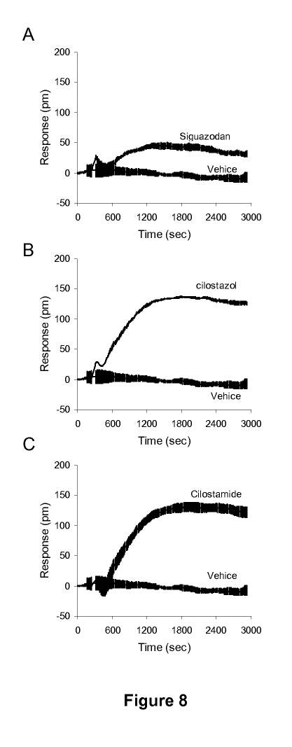

11. FIG. 8 shows examples of the PDE3 inhibitors-induced DMR signals of synchronized A431 cells: (A) siguazodan, (B) cilostazol, and (C) cilostamide, in comparison with the DMR signals when the cells were treated with the vehicle only (i.e., the HBSS buffer). The concentrations of all inhibitors were at 12.5 micromolar. The A431 cells were synchronized using the standard protocol, same as indicated in FIG. 6.

12. FIG. 9 shows examples of the PDE3 specific inhibitors-induced DMR signals of synchronized A431 cells: (A) milrinone, (B) anagrelide, in comparison with the DMR signals when the cells were treated with the vehicle only (i.e., the HBSS buffer). The concentrations of all inhibitors were at 12.5 micromolar. The A431 cells were synchronized using the standard protocol, same as indicated in FIG. 6.

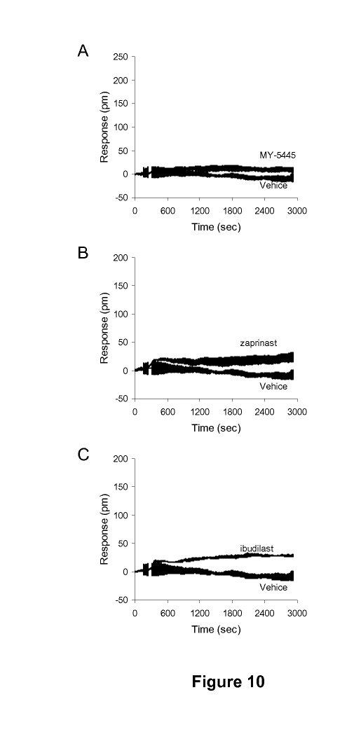

13. FIG. 10 shows examples of the PDES specific inhibitors-induced DMR signals of synchronized A431 cells: (A) MY-5445, (B) Zaprinast, (C) ibudilast, in comparison with the DMR signals when the cells were treated with the vehicle only (i.e., the HBSS buffer). The concentrations of all inhibitors were at 12.5 micromolar. The A431 cells were synchronized using the standard protocol, same as indicated in FIG. 6.

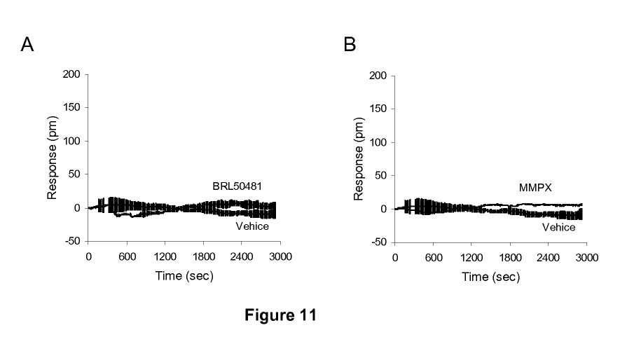

14. FIG. 11 shows examples of (A) the PDE7 specific inhibitor BRL50481, and (B) the PDE1 specific inhibitor MMPX-induced DMR signals of synchronized A431 cells, in comparison with the DMR signals when the cells were treated with the vehicle only (i.e., the HBSS buffer). The concentrations of all inhibitors were at 12.5 micromolar. The A431 cells were synchronized using the standard protocol, same as indicated in FIG. 6.

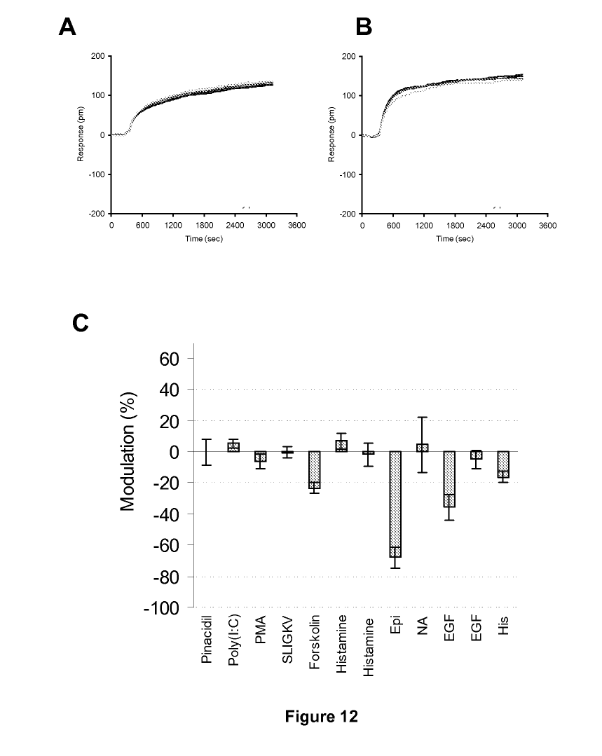

15. FIG. 12 shows an example of molecular biosensor index for tyrphostin 51, which include the primary DMR profile of tyrphostin 51 in quiescent A431 cells (A), and A549 cells (B), and the modulation index of tyrphostins 51 against a panel of markers across the two distinct cell lines (C).

### V. DETAILED DESCRIPTION OF THE INVENTION

16. Various embodiments of the disclosure will be described in detail with reference to drawings, if any. Reference to various embodiments does not limit the scope of the disclosure, which is limited only by the scope of the claims attached hereto. Additionally, any examples set forth in this specification are not intended to be limiting and merely set forth some of the many possible embodiments for the claimed invention.

### A. DEFINITIONS

### 1. A

17. As used in the specification and the appended claims, the singular forms “a,” “an” and “the” or like terms include plural referents unless the context clearly dictates otherwise. Thus, for example, reference to “a PDE inhibitor” includes mixtures of two or more such inhibitors, and the like.

### 2. Abbreviations

18. Abbreviations, which are well known to one of ordinary skill in the art, may be used (e.g., “h” or “hr” for hour or hours, “g” or “gm” for gram(s), “mL” for milliliters, and “rt” for room temperature, “nm” for nanometers, “M” for molar, and like abbreviations).

### 3. About

19. About modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

### 4. “Another Period of Time”

20. An “another period of time” or “extended period of time” or like terms is a period of time sequentially occurring after a period of time or after a treatment. The time period can vary greatly, from 10 min to 1 hr, 2 hrs, 4 hrs, 8 hrs, or 24 hrs.

### 5. Assaying

21. Assaying, assay, or like terms refers to an analysis to determine a characteristic of a substance, such as a molecule or a cell, such as for example, the presence, absence, quantity, extent, kinetics, dynamics, or type of an a cell's optical or bioimpedance response upon stimulation with one or more exogenous stimuli, such as a ligand or marker. Producing a biosensor signal of a cell's response to a stimulus can be an assay.

### 6. Assaying the Response

22. “Assaying the response” or like terms means using a means to characterize the response. For example, if a molecule is brought into contact with a cell, a biosensor can be used to assay the response of the cell upon exposure to the molecule.

### 7. Attach

23. “Attach,” “attachment,” “adhere,” “adhered,” “adherent,” “immobilized”, or like terms generally refer to immobilizing or fixing, for example, a surface modifier substance, a compatibilizer, a cell, a ligand candidate molecule, and like entities of the disclosure, to a surface, such as by physical absorption, chemical bonding, and like processes, or combinations thereof. Particularly, “cell attachment,” “cell adhesion,” or like terms refer to the interacting or binding of cells to a surface, such as by culturing, or interacting with cell anchoring materials, compatibilizer (e.g., fibronectin, collagen, lamin, gelatin, polylysine, etc.), or both. “Adherent cells,” “immobilized cells”, or like terms refer to a cell or a cell line or a cell system, such as a prokaryotic or eukaryotic cell, that remains associated with, immobilized on, or in certain contact with the outer surface of a substrate. Such types of cells after culturing can withstand or survive washing and medium exchanging processes staying adhered, a process that is prerequisite to many cell-based assays.

### 8. Biosensor

24. Biosensor or like terms refer to a device for the detection of an analyte that combines a biological component with a physicochemical detector component. The biosensor typically consists of three parts: a biological component or element (such as tissue, microorganism, pathogen, cells, or combinations thereof), a detector element (works in a physicochemical way such as optical, piezoelectric, electrochemical, thermometric, or magnetic), and a transducer associated with both components. The biological component or element can be, for example, a living cell, a pathogen, or combinations thereof. In embodiments, an optical biosensor can comprise an optical transducer for converting a molecular recognition or molecular stimulation event in a living cell, a pathogen, or combinations thereof into a quantifiable signal.

### 9. Biosensor Index

25. A “biosensor index” or like terms is an index made up of a collection of biosensor data. A biosensor index can be a collection of biosensor profiles, such as primary profiles, or secondary profiles. The index can be comprised of any type of data. For example, an index of profiles could be comprised of just an N-DMR data point, it could be a P-DMR data point, or both or it could be an impedence data point. It could be all of the data points associated with the profile curve.

### 10. Biosensor Response

26. A “biosensor response”, “biosensor output signal”, “biosensor signal” or like terms is any reaction of a sensor system having a cell to a cellular response. A biosensor converts a cellular response to a quantifiable sensor response. A biosensor response is an optical response upon stimulation as measured by an optical biosensor such as RWG or SPR or it is a bioimpedence response of the cells upon stimulation as measured by an electric biosensor. Since a biosensor response is directly associated with the cellular response upon stimulation, the biosensor response and the cellular response can be used interchangeably, in embodiments of disclosure.

### 11. Biosensor Signal

27. A “biosensor signal” or like terms refers to the signal of cells measured with a biosensor that is produced by the response of a cell upon stimulation.

### 12. Biosensor Surface

28. A biosensor surface or like words is any surface of a biosensor which can have a cell cultured on it. The biosensor surface can be tissue culture treated, or extracellular matrix material (e.g., fibronectin, laminin, collagen, or the like) coated, or synthetic material (e.g, poly-lysine) coated.

### 13. Carbonic Anahydrase Inhibitor

A carbonic anahydrase inhibitor is any molecule, compound, or composition that suppress the activity of carbonic anhydrase. Carbonic anhydrases (or carbonate dehydratases) are a family of enzymes that catalyze the rapid conversion of carbon dioxide to bicarbonate and protons. The active site of most carbonic anhydrases contains a zinc ion; they are therefore classified as metalloenzymes. Carbonic anhydrase inhibitors include, but not limited to, acetazolamide, methazolamide, dorzolamide, and topiramate.

### 14. Cell

29. Cell or like term refers to a small usually microscopic mass of protoplasm bounded externally by a semipermeable membrane, optionally including one or more nuclei and various other organelles, capable alone or interacting with other like masses of performing all the fundamental functions of life, and forming the smallest structural unit of living matter capable of functioning independently including synthetic cell constructs, cell model systems, and like artificial cellular systems.

30. A cell can include different cell types, such as a cell associated with a specific disease, a type of cell from a specific origin, a type of cell associated with a specific target, or a type of cell associated with a specific physiological function. A cell can also be a native cell, an engineered cell, a transformed cell, an immortalized cell, a primary cell, an embryonic stem cell, an adult stem cell, a cancer stem cell, or a stem cell derived cell.

31. Human consists of about 210 known distinct cell types. The numbers of types of cells can almost unlimited, considering how the cells are prepared (e.g., engineered, transformed, immortalized, or freshly isolated from a human body) and where the cells are obtained (e.g., human bodies of different ages or different disease stages, etc).

### 15. Cell Culture

32. “Cell culture” or “cell culturing” refers to the process by which either prokaryotic or eukaryotic cells are grown under controlled conditions. “Cell culture” not only refers to the culturing of cells derived from multicellular eukaryotes, especially animal cells, but also the culturing of complex tissues and organs.

### 16. Cell Panel

33. A “cell panel” or like terms is a panel which comprises at least two types of cells. The cells can be of any type or combination disclosed herein.

### 17. Cellular Background

A “cellular background” or like terms is a type of cell having a specific state. For example, different types of cells have different cellular backgrounds (e.g., differential expression or organization of cellular receptors). A same type of cell but having different states also has different cellular backgrounds. The different states of the same type of cells can be achieved through culture (e.g., cell cycle arrested, or proliferating or quiescent states), or treatment (e.g., different pharmacological agent-treated cells).

### 18. Cellular Process

34. A cellular process or like terms is a process that takes place in or by a cell. Examples of cellular process include, but not limited to, proliferation, apoptosis, necrosis, differentiation, cell signal transduction, polarity change, migration, or transformation.

### 19. Cellular Response

35. A “cellular response” or like terms is any reaction by the cell to a stimulation.

### 20. Cellular Target

36. A “cellular target” or like terms is a biopolymer such as a protein or nucleic acid whose activity can be modified by an external stimulus. Cellular targets are most commonly proteins such as enzymes, kinases, ion channels, and receptors.

### 21. Components

37. Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these molecules may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### 22. Compounds and Compositions

38. Compounds and compositions have their standard meaning in the art. It is understood that wherever, a particular designation, such as a molecule, substance, marker, cell, or reagent compositions comprising, consisting of, and consisting essentially of these designations are disclosed. Thus, where the particular designation marker is used, it is understood that also disclosed would be compositions comprising that marker, consisting of that marker, or consisting essentially of that marker. Where appropriate wherever a particular designation is made, it is understood that the compound of that designation is also disclosed. For example, if particular biological material, such as a PDE4 inhibitor, is disclosed, the PDE4 inhibitor in its compound form is also disclosed.

### 23. Comprise

39. Throughout the description and claims of this specification, the word “comprise” and variations of the word, such as “comprising” and “comprises,” means “including but not limited to,” and is not intended to exclude, for example, other additives, components, integers or steps.

### 24. Consisting Essentially of

40. “Consisting essentially of” in embodiments refers to, for example, a surface composition, a method of making or using a surface composition, formulation, or composition on the surface of the biosensor, and articles, devices, or apparatus of the disclosure, and can include the components or steps listed in the claim, plus other components or steps that do not materially affect the basic and novel properties of the compositions, articles, apparatus, and methods of making and use of the disclosure, such as particular reactants, particular additives or ingredients, a particular agents, a particular cell or cell line, a particular surface modifier or condition, a particular ligand candidate, or like structure, material, or process variable selected. Items that may materially affect the basic properties of the components or steps of the disclosure or may impart undesirable characteristics to the present disclosure include, for example, decreased affinity of the cell for the biosensor surface, aberrant affinity of a stimulus for a cell surface receptor or for an intracellular receptor, anomalous or contrary cell activity in response to a ligand candidate or like stimulus, and like characteristics.

### 25. Characterizing

41. Characterizing or like terms refers to gathering information about any property of a substance, such as a ligand, molecule, marker, or cell, such as obtaining a profile for the ligand, molecule, marker, or cell.

### 26. Contacting

42. Contacting or like terms means bringing into proximity such that a molecular interaction can take place, if a molecular interaction is possible between at least two things, such as molecules, cells, markers, at least a compound or composition, or at least two compositions, or any of these with an article(s) or with a machine. For example, contacting refers to bringing at least two compositions, molecules, articles, or things into contact, i.e. such that they are in proximity to mix or touch. For example, having a solution of composition A and cultured cell B and pouring solution of composition A over cultured cell B would be bringing solution of composition A in contact with cell culture B. Contacting a cell with a ligand would be bringing a ligand to the cell to ensure the cell have access to the ligand.

43. It is understood that anything disclosed herein can be brought into contact with anything else. For example, a cell can be brought into contact with a marker or a molecule, a biosensor, and so forth.

### 27. Control

44. The terms control or “control levels” or “control cells” or like terms are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard. For example, a control can refer to the results from an experiment in which the subjects or objects or reagents etc are treated as in a parallel experiment except for omission of the procedure or agent or variable etc under test and which is used as a standard of comparison in judging experimental effects. Thus, the control can be used to determine the effects related to the procedure or agent or variable etc. For example, if the effect of a test molecule on a cell was in question, one could a) simply record the characteristics of the cell in the presence of the molecule, b) perform a and then also record the effects of adding a control molecule with a known activity or lack of activity, or a control composition (e.g., the assay buffer solution (the vehicle)) and then compare effects of the test molecule to the control. In certain circumstances once a control is performed the control can be used as a standard, in which the control experiment does not have to be performed again and in other circumstances the control experiment should be run in parallel each time a comparison will be made.

### 28. Defined Pathway(s)

45. A “defined pathway” or like terms is a specific pathway, such as Gq pathway, Gs pathway, Gi pathway, EGFR (epidermal growth factor receptor) pathway, or PKC (protein kinase C) pathway.

### 29. Detect

46. Detect or like terms refer to an ability of the apparatus and methods of the disclosure to discover or sense a molecule-induced cellular response and to distinguish the sensed responses for distinct molecules.

### 30. Direct Action (of a Drug Candidate Molecule)

47. A “direct action” or like terms is a result (of a drug candidate molecule”) acting on a cell.

### 31. DMR Index

48. A “DMR index” or like terms is a biosensor index made up of a collection of DMR data.

### 32. DMR Response

49. A “DMR response” or like terms is a biosensor response using an optical biosensor. The DMR refers to dynamic mass redistribution or dynamic cellular matter redistribution. A P-DMR is a positive DMR response, a N-DMR is a negative DMR response, and a RP-DMR is a recovery P-DMR response.

### 33. DMR Signal

50. A “DMR signal” or like terms refers to the signal of cells measured with an optical biosensor that is produced by the response of a cell upon stimulation.

### 34. Drug Candidate Molecule

51. A drug candidate molecule or like terms is a test molecule which is being tested for its ability to function as a drug or a pharmacophore. This molecule may be considered as a lead molecule.

### 35. Early Culture

52. An early culture or like terms is the relative status of cells during a culture which is often related to its confluency or cell cycle states Early culture is cell culture towards high confluency, greater than or equal to 90%. Time less than or equal to the cell doubling time.

### 36. Efficacy

53. Efficacy or like terms is the capacity to produce a desired size of an effect under ideal or optimal conditions. It is these conditions that distinguish efficacy from the related concept of effectiveness, which relates to change under real-life conditions. Efficacy is the relationship between receptor occupancy and the ability to initiate a response at the molecular, cellular, tissue or system level.

### 37. High Confluency

54. Cell confluency or like terms refers to the coverage or proliferation that the cells are allowed over or throughout the culture medium. Since many types of cells can undergo cell contact inhibition, a high confluency means that the cells cultured reach high coverage (>90%) on a tissue culture surface or a biosensor surface, and have significant restriction to the growth of the cells in the medium. Conversely, a low confluency (e.g., a confluency of 40-60%) means that there may be little or no restriction to the growth of the cells in/on the medium and they can be assumed to be in a growth phase.

### 38. Higher and Inhibit and Like Words

55. The terms higher, increases, elevates, or elevation or like terms or variants of these terms, refer to increases above basal levels, e.g., as compared a control. The terms low, lower, reduces, decreases or reduction or like terms or variation of these terms, refer to decreases below basal levels, e.g., as compared to a control. For example, basal levels are normal in vivo levels prior to, or in the absence of, or addition of a molecule such as an agonist or antagonist to a cell. Inhibit or forms of inhibit or like terms refers to reducing or suppressing.

### 39. “In the Presence of the Molecule”

56. “in the presence of the molecule” or like terms refers to the contact or exposure of the cultured cell with the molecule. The contact or exposure can take place before, or at the time, the stimulus is brought to contact with the cell.

### 40. Index

57. An index or like terms is a collection of data. For example, an index can be a list, table, file, or catalog that contains one or more modulation profiles. It is understood that an index can be produced from any combination of data. For example, a DMR profile can have a P-DMR, a N-DMR, and a RP-DMR. An index can be produced using the completed date of the profile, the P-DMR data, the N-DMR data, the RP-DMR data, or any point within these, or in combination of these or other data. The index is the collection of any such information. Typically, when comparing indexes, the indexes are of like data, i.e. P-DMR to P-DMR data.

### 41. “Indicator for the Mode of Action of the Molecule”

58. An “indicator” or like terms is a thing that indicates. Specifically, “an indicator for the mode of action of the molecule” means a thing, such as the similarity of biosensor index of a molecule in comparison with a biosensor index of a well-known modulator, that can be interpreted that the molecule and the well-known modulator share similar mode of action.

### 42. Known Modulator

59. A known modulator or like terms is a modulator where at least one of the targets is known with a known affinity. For example, a known modulator could be a PDE4 inhibitor.

### 43. Known Modulator DMR Index

60. A “known modulator DMR index” or like terms is a modulator DMR index produced by data collected for a known modulator. For example, a known modulator DMR index can be made up of a profile of the known modulator acting on the panel of cells, and the modulation profile of the known modulator against the panels of markers, each panel of markers for a cell in the panel of cells.

### 44. Known Modulator Biosensor Index

61. A “known modulator biosensor index” or like terms is a modulator biosensor index produced by data collected for a known modulator. For example, a known modulator biosensor index can be made up of a profile of the known modulator acting on the panel of cells, and the modulation profile of the known modulator against the panels of markers, each panel of markers for a cell in the panel of cells.

### 45. Known Molecule

62. A known molecule or like terms is a molecule with known pharmacological/biological/physiological/pathophysiological activity whose precise mode of action(s) may be known or unknown.

### 46. Library

63. A library or like terms is a collection. The library can be a collection of anything disclosed herein. For example, it can be a collection, of indexes, an index library; it can be a collection of profiles, a profile library; or it can be a collection of DMR indexes, a DMR index library; Also, it can be a collection of molecules, a molecule library; it can be a collection of cells, a cell library; it can be a collection of markers, a marker library; A library can be for example, random or non-random, determined or undetermined. For example, disclosed are libraries of DMR indexes or biosensor indexes of known modulators.

### 47. Ligand

64. A ligand or like terms is a substance or a composition or a molecule that is able to bind to and form a complex with a biomolecule to serve a biological purpose. Actual irreversible covalent binding between a ligand and its target molecule is rare in biological systems. Ligand binding to receptors alters the chemical conformation, i.e., the three dimensional shape of the receptor protein. The conformational state of a receptor protein determines the functional state of the receptor. The tendency or strength of binding is called affinity. Ligands include substrates, blockers, inhibitors, activators, and neurotransmitters. Radioligands are radioisotope labeled ligands, while fluorescent ligands are fluorescently tagged ligands; both can be considered as ligands are often used as tracers for receptor biology and biochemistry studies. Ligand and modulator are used interchangeably.

### 48. Low CO₂ Environment

65. A low CO₂ environment is an environment that has less than 4.5% CO₂.

### 49. Material

66. Material is the tangible part of something (chemical, biochemical, biological, or mixed) that goes into the makeup of a physical object.

### 50. Medium

67. A medium is any mixture within which cells can be cultured. A growth medium is an object in which microorganisms or cells experience growth.

### 51. Modulate

68. To modulate, or forms thereof, means either increasing, decreasing, or maintaining a cellular activity mediated through a cellular target. It is understood that wherever one of these words is used it is also disclosed that it could be 1%, 5%, 10%, 20%, 50%, 100%, 500%, or 1000% increased from a control, or it could be 1%, 5%, 10%, 20%, 50%, or 100% decreased from a control.

### 52. Modulate the DMR Signal

69. “Modulate the DMR signal or like terms is to cause changes of the DMR signal or profile of a cell in response to stimulation with a molecule.

### 53. Modulator

70. A modulator or like terms is a molecule, such as a ligand, that controls the activity of a cellular target. It is a signal modulating molecule binding to a cellular target, such as a target protein.

### 54. Modulator Biosensor Index

71. A “modulator biosensor index” or like terms is a biosensor index produced by data collected for a modulator, such as DMR data. For example, a modulator biosensor index can be made up of a profile of the modulator acting on the panel of cells.

### 55. Molecule

72. As used herein, the terms “molecule” or like terms refers to a biological or biochemical or chemical entity that exists in the form of a chemical molecule or molecule with a definite molecular weight. A molecule or like terms is a chemical, biochemical or biological molecule, regardless of its size.

73. Many molecules are of the type referred to as organic molecules (molecules containing carbon atoms, among others, connected by covalent bonds), although some molecules do not contain carbon (including simple molecular gases such as molecular oxygen and more complex molecules such as some sulfur-based polymers). The general term “molecule” includes numerous descriptive classes or groups of molecules, such as proteins, nucleic acids, carbohydrates, steroids, organic pharmaceuticals, small molecule, receptors, antibodies, and lipids. When appropriate, one or more of these more descriptive terms (many of which, such as “protein,” themselves describe overlapping groups of molecules) will be used herein because of application of the method to a subgroup of molecules, without detracting from the intent to have such molecules be representative of both the general class “molecules” and the named subclass, such as proteins. Unless specifically indicated, the word “molecule” would include the specific molecule and salts thereof, such as pharmaceutically acceptable salts.

### 56. Molecule Index

74. A “molecule index” or like terms is an index related to the molecule.

### 57. Molecule Mixture

75. A molecule mixture or like terms is a mixture containing at least two molecules. The two molecules can be, but not limited to, structurally different (i.e., enantiomers), or compositionally different (e.g., protein isoforms, glycoform, or an antibody with different poly(ethylene glycol) (PEG) modifications), or structurally and compositionally different (e.g., unpurified natural extracts, or unpurified synthetic compounds).

### 58. Molecule-Treated Cell

76. A molecule-treated cell or like terms is a cell that has been exposed to a molecule.

### 59. Native Cell

77. A native cell is any cell that has not been genetically engineered. A native cell can be a primary cell, a immortalized cell, a transformed cell line, a stem cell, or a stem cell derived cell.

### 60. Normalizing

78. Normalizing or like terms means, adjusting data, or a profile, or a response, for example, to remove at least one common variable.

### 61. Optional

79. “Optional” or “optionally” or like terms means that the subsequently described event or circumstance can or cannot occur, and that the description includes instances where the event or circumstance occurs and instances where it does not. For example, the phrase “optionally the composition can comprise a combination” means that the composition may comprise a combination of different molecules or may not include a combination such that the description includes both the combination and the absence of the combination (i.e., individual members of the combination).

### 62. Or

80. The word “or” or like terms as used herein means any one member of a particular list and also includes any combination of members of that list.

### 63. Panel

81. A panel or like terms is a predetermined set of specimens (cells, or pathways). A panel can be produced from picking specimens from a library.

### 64. pH Buffered Assay Solution

82. A pH buffered assay solution is any solution which has been buffered to have a physiological pH (typically pH of 7.1).

### 65. Panning

83. Panning or like terms refers to screening a cell or cells for the presence of one or more receptors or cellular targets.

### 66. PDE Inhibitor

84. A PDE inhibitor or like words is any molecule, compound, or composition, that inhibits or supresses the activity of a PDE.

### 67. PDE Modulator

85. A PDE modulator or like words is any molecule, compound, or composition, that modulates the activity of a PDE.

### 68. PDE Activator

86. A PDE inhibitor or like words is any molecule, compound, or composition, that activates the activity of a PDE.

### 69. “Period of Time”

87. A “period of time” refers to any period representing a passage of time. For example, 1 second, 1 minute, 1 hour, 1 day, and 1 week are all periods of time.

### 70. Positive Control

88. A “positive control” or like terms is a control that shows that the conditions for data collection can lead to data collection.

### 71. Potency

89. Potency or like terms is a measure of molecule activity expressed in terms of the amount required to produce an effect of given intensity. The potency is proportional to affinity and efficacy. Affinity is the ability of the drug molecule to bind to a receptor.

### 72. Primary Profile

90. A “primary profile” or like terms refers to a biosensor response or biosensor output signal or profile which is produced when a molecule contacts a cell. Typically, the primary profile is obtained after normalization of initial cellular response to the net-zero biosensor signal (i.e., baseline)

### 73. Profile

91. A profile or like terms refers to the data which is collected for a composition, such as a cell. A profile can be collected from a label free biosensor as described herein.

### 74. Publications

92. Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### 75. Pulse Stimulation Assay

93. A “pulse stimulation assay” or like terms can used, wherein the cell is only exposed to a molecule for a very short of time (e.g., seconds, or several minutes). This pulse stimulation assay can be used to study the kinetics of the molecule acting on the cells/targets, as well as its impact on the marker-induced biosensor signals. The pulse stimulation assay can be carried out by simply replacing the molecule solution with the cell assay buffer solution by liquid handling device at a given time right after the molecule addition.

### 76. Quiescence

94. Quiescence or the like terms refers to a state of being quiet, still, at rest, dormant, inactive. Quiescence may refer to the G₀ phase of a cell in the cell cycle; or quiescence is the state of a cell when it is not dividing. Cellular quiescence is defined as reversible growth/proliferation arrest induced by diverse anti-mitogenic signals, e.g., mitogen (e.g., growth factor) withdrawal, contact inhibition, and loss of adhesion.

### 77. Ranges

95. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

### 78. Receptor

96. A receptor or like terms is a protein molecule embedded in either the plasma membrane or cytoplasm of a cell, to which a mobile signaling (or “signal”) molecule may attach. A molecule which binds to a receptor is called a “ligand,” and may be a peptide (such as a neurotransmitter), a hormone, a pharmaceutical drug, or a toxin, and when such binding occurs, the receptor goes into a conformational change which ordinarily initiates a cellular response. However, some ligands merely block receptors without inducing any response (e.g. antagonists). Ligand-induced changes in receptors result in physiological changes which constitute the biological activity of the ligands.

### 79. Response

97. A response or like terms is any reaction to any stimulation.

### 80. “Robust Biosensor Signal”

98. A “robust biosensor signal” is a biosensor signal whose amplitude(s) is significantly (such as 3×, 10×, 20×, 100×, or 1000×) above either the noise level, or the negative control response. The negative control response is often the biosensor response of cells after addition of the assay buffer solution (i.e., the vehicle). The noise level is the biosensor signal of cells without further addition of any solution. It is worth noting that the cells are always covered with a solution before addition of any solution.

### 81. “Robust DMR Signal”

99. A “robust DMR signal” or like terms is a DMR form of a “robust biosensor signal.”

### 82. Sample

100. By sample or like terms is meant an animal, a plant, a fungus, etc.; a natural product, a natural product extract, etc.; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

### 83. Serum Containing Medium

101. Serum containing medium or like words is any cell culture medium which contains serum (such as fetal bovine serum). Fetal bovine serum (or fetal calf serum) is the portion of plasma remaining after coagulation of blood, during which process the plasma protein fibrinogen is converted to fibrin and remains behind in the clot. Fetal Bovine serum comes from the blood drawn from the unborn bovine fetus via a closed system venipuncture at the abattoir. Fetal Bovine Serum (FBS) is the most widely used serum due to being low in antibodies and containing more growth factors, allowing for versatility in many different applications. FBS is used in the culturing of eukaryotic cells.

### 84. Serum Depleted Medium

102. A serum depleted medium is any cell culture medium that does not contain serum.

### 85. “Short Period of Time”

103. A “short period of time” or like terms is a time period that is typically shorter than the duplication of cells under standard culture.

### 86. Signaling Pathway(s)

104. A “defined pathway” or like terms is a path of a cell from receiving a signal (e.g., an exogenous ligand) to a cellular response (e.g., increased expression of a cellular target). In some cases, receptor activation caused by ligand binding to a receptor is directly coupled to the cell's response to the ligand. For example, the neurotransmitter GABA can activate a cell surface receptor that is part of an ion channel. GABA binding to a GABA A receptor on a neuron opens a chloride-selective ion channel that is part of the receptor. GABA A receptor activation allows negatively charged chloride ions to move into the neuron which inhibits the ability of the neuron to produce action potentials. However, for many cell surface receptors, ligand-receptor interactions are not directly linked to the cell's response. The activated receptor must first interact with other proteins inside the cell before the ultimate physiological effect of the ligand on the cell's behavior is produced. Often, the behavior of a chain of several interacting cell proteins is altered following receptor activation. The entire set of cell changes induced by receptor activation is called a signal transduction mechanism or pathway. The signaling pathway can be either relatively simple or quite complicated.

### 87. Similarity and Similarity of Indexes

105. “Similarity of indexes” or like terms is a term to express the similarity between two indexes, or among at least three indices, one for a molecule, based on the patterns of indices, and/or a matrix of scores. The matrix of scores are strongly related to their counterparts, such as the signatures of the primary profiles of different molecules in corresponding cells, and the nature and percentages of the modulation profiles of different molecules against a marker. For example, higher scores are given to more-similar characters, and lower or negative scores for dissimilar characters. Because there are only three types of modulation, positive, negative and neutral, found in the molecule modulation index, the similarity matrices are relatively simple. For example, a simple matrix will assign a positive modulation a score of +1, a negative modulator a score of −1, and a neutral modulation a score of 0.

106. Alternatively, different scores can be given for a type of modulation but with different scales. For example, a positive modulation of 10%, 20%, 30%, 40%, 50%, 60%, 100%, 200%, etc, can be given a score of +1, +2, +3, +4, +5, +6, +10, +20, correspondingly. Conversely, for negative modulation, similar but in opposite score can be given. Following this approach, the modulation index of tyrphostin 51 against panels of markers, as shown in FIG. 10C, illustrates that the known EGFR inhibitor tyrphostins 51 modulates differently the biosensor responses induced by different markers: pinacidil (0%), poly(I:C) (+5%), PMA (−6%), SLIGKV-amide (0%), forskolin (−23%), histamine (+6% the histamine early response; and 0% the histamine late response), all in A549 cell; and epinephrine (−68%), nicotinic acid (+4%), EGF (P-DMR, −36%), EGF (N-DMR, −5%), and histamine (−16%), all in quiescent A431 cells. Thus, the score of HA1077 modulation index in coordination can be assigned as (0, 0.5, −0.6, 0, −2.3, 0.6, 0, −6.8, 0.4, −3.6, −0.5, −1.6). Once a molecular index is generated, the molecular index can be compared with a library of known modulators to determine the mode(s) of action of the molecule of interest. From the biosensor index of tryphostin 51, one can conclude that tyrphostins 51 displays polypharmacology, since it acts as an EGFR inhibitor (inhibiting the EGF induced DMR signal in A431), and also a PDE4 inhibitor (inhibiting both epinephrine and histamine responses in A431, as well as the forskolin response in A549).

### 88. Starving the Cells

107. Starving the cells or like terms refers to a process to drive cells into quiescence during cell culture. The mitogen (e.g., serum or growth factors) withdrawl from the cell culture medium during the cell culture is the most common means to starving the cells. The mitogen withdrawl may be used in conjunction with other means (e.g., contact inhibition).

### 89. Substance

108. A substance or like terms is any physical object. A material is a substance. Molecules, ligands, markers, cells, proteins, and DNA can be considered substances. A machine or an article would be considered to be made of substances, rather than considered a substance themselves.

### 90. Synchronized Cells

109. Synchonized cells or the like terms refer to a population of cells wherein the majority of cells in a single well of a microtiter plate are in the same state (e.g., the same cell cycle (such as G₀ or G₂)). Synchronize(d) cells or the like term can also refer to the manipulation of the environment surrounding the cells or the conditions at which cells are grown which results in a population of cells wherein most cells are in the same stage of the cell cycle.

### 91. Stable

110. When used with respect to pharmaceutical compositions, the term “stable” or like terms is generally understood in the art as meaning less than a certain amount, usually 10%, loss of the active ingredient under specified storage conditions for a stated period of time. The time required for a composition to be considered stable is relative to the use of each product and is dictated by the commercial practicalities of producing the product, holding it for quality control and inspection, shipping it to a wholesaler or direct to a customer where it is held again in storage before its eventual use. Including a safety factor of a few months time, the minimum product life for pharmaceuticals is usually one year, and preferably more than 18 months. As used herein, the term “stable” references these market realities and the ability to store and transport the product at readily attainable environmental conditions such as refrigerated conditions, 2° C. to 8° C.

### 92. Subject

111. As used throughout, by a subject or like terms is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) and mammals, non-human mammals, primates, non-human primates, rodents, birds, reptiles, amphibians, fish, and any other animal. In one aspect, the subject is a mammal such as a primate or a human. The subject can be a non-human.

### 93. Suspension Cells

112. “Suspension cells” refers to a cell or a cell line that is preferably cultured in a medium wherein the cells do not attach or adhere to the surface of a substrate during the culture. However, suspension cells can, in general, be brought to contact with the biosensor surface, by either chemical (e.g., covalent attachment, or antibody-cell surface receptor interactions), or physical means (e.g., settlement down, due to the gravity force, the bottom of a well wherein a biosensor is embedded). Thus, suspension cells can also be used for biosensor cellular assays.

### 94. Test Molecule

113. A test molecule or like terms is a molecule which is used in a method to gain some information about the test molecule. A test molecule can be an unknown or a known molecule.

### 95. Treating

114. Treating or treatment or like terms can be used in at least two ways. First, treating or treatment or like terms can refer to administration or action taken towards a subject, manipulating a subject. Second, treating or treatment or like terms can refer to mixing any two things together, such as any two or more substances together, such as a molecule and a cell. This mixing will bring the at least two substances together such that a contact between them can take place. For instance, “treating cell to reach high confluency”, means to take care or manipulate cells so they reach high confluency.

115. When treating or treatment or like terms is used in the context of a subject with a disease, it does not imply a cure or even a reduction of a symptom for example. When the term therapeutic or like terms is used in conjunction with treating or treatment or like terms, it means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

### 96. Trigger

116. A trigger or like terms refers to the act of setting off or initiating an event, such as a response.

### 97. Ultra High Confluency

117. Ultra high confluency or the like terms refers to a population of cells that have at least 99% confluency in the end of cell culture.

### 98. Unknown Molecule

118. An unknown molecule or like terms is a molecule with unknown biological/pharmacological/physiological/pathophysiological activity.

### 99. Values

119. Specific and preferred values disclosed for components, ingredients, additives, cell types, markers, and like aspects, and ranges thereof, are for illustration only; they do not exclude other defined values or other values within defined ranges. The compositions, apparatus, and methods of the disclosure include those having any value or any combination of the values, specific values, more specific values, and preferred values described herein.

120. Thus, the disclosed methods, compositions, articles, and machines, can be combined in a manner to comprise, consist of, or consist essentially of, the various components, steps, molecules, and composition, and the like, discussed herein. They can be used, for example, in methods for characterizing a molecule including a ligand as defined herein; a method of producing an index as defined herein; or a method of drug discovery as defined herein.

### 100. Weakly Adherent Cells

121. “Weakly adherent cells” refers to a cell or a cell line or a cell system, such as a prokaryotic or eukaryotic cell, which weakly interacts, or associates or contacts with the surface of a substrate during cell culture. However, these types of cells, for example, human embryonic kidney (HEK) cells, dissociate from the surface of a substrate by the physically disturbing approach of washing or medium exchange.

### B. PDES (PHOSPHODIESTERASES)

122. The cyclic nucleotide phosphodiesterases (PDE) comprise a group of enzymes that degrade the phosphodiester bond in the second messenger molecules cAMP and cGMP. They regulate the localization, duration, and amplitude of cyclic nucleotide signaling within subcellular domains. PDEs are therefore important regulators of signal transduction mediated by these second messenger molecules. There are 11 families of PDEs from 21 different genes. Each PDE family is distinguished functionally by unique enzymatic characteristics and pharmacological profiles as well as distinct tissue distribution and cellular expression patterns. Because PDEs regulate a variety of cellular functions, they have become important drug targets for the treatment of several diseases including sexual dysfunction, asthma, chronic obstructive pulmonary disease, neurodegenerative diseases (Parkinson's disease and Alzheimer's), diabetes, vascular diseases, osteoporosis, cancer, and rheumatoid arthritis.

123. PDE4 isoenzymes specifically hydrolyze cAMP and are therapeutic targets for the treatment of several inflammatory disorders. A number of biochemical assays are available for screening of PDEs that use purified recombinant PDE enzymes and cAMP or cGMP as the substrate. However, a cell-free assay environment may not faithfully reproduce the physiological environment of the cell (i.e. due to differences in buffer components, pH, and cofactors etc). In addition, molecules active in enzyme assays are often inactive in disease models due to poor cell membrane permeability, intracellular metabolism, or active sites with highly polar groups. Therefore, inhibitors identified from enzyme assays usually need to be optimized in cell-based assays before proceeding to animal model studies.

124. Cell-based PDE assays have also been demonstrated. In a typical radioimmunoassay, PDE activity is often measured using radiolabels after the cells, having transitly or stably expressed a PDE, are lysed. These cell-based PDE4 assays have a complicated assay procedure and they have relatively limited screening throughput, in addition to being invasive and destructive. Alternatively, a cell-based luciferase reporter gene assay using the cAMP responsive element (CRE) binding sequence was reported for the measurement of PDE4, PDE7, and PDE10 activities (Bora, R. S., et al., A reporter gene assay for screening of PDE4 subtype selective inhibitors. Biochemical and Biophysical Research Communications, 2007, 356, 153-158; Bora, R. S., et al., Development of a cell-based assay for screening of phosphodiesterase 10A (PDE10A) inhibitors using a stable recombinant HEK-293 cell line expressing high levels of PDE10A. Biotechnology and Applied Biochemistry, 2008, 49, 129-134.). However, reporter gene assays often cause shifts in measured compound activities and produce increased false positives in compound library screening due to a long cascade of signal transduction and reporter gene transcription and translation. Finally, cell-based PDE4 assays using a cyclic nucleotide-gated (CNG) cation channel as a biosensor have been reported (Titus, S. A., et al., A cell-based PDE4 assay in 1536-well plate format for high-throughput screening. Journal of Biomolecular Screening 2008, 13, 609-618). Here, PDE activity is detected using the calcium dye Fura-2, or voltage-clamping, or membrane potential sensitive fluorescence measures. However, these methods require engineered cells coexpressing a constitutively active Gₛ-coupled receptor as a driving force for cAMP production together with a CNG (cyclic nucleotide gated ion channel) channel as a cAMP sensor. The constitutive activity of transfected receptors in the PDE4 cell line maintains a moderate level of cAMP production. The continual cAMP production provides the basis for the measurement of PDE activity. This cell-based PDE4 assay uses a stably transfected CNG cation channel as a biosensor whose signal can be detected by a change in membrane potential. In the absence of a PDE inhibitor, the moderate level of cAMP in the cell line having an overexpressed constitutively active Gs-coupled receptor is rapidly hydrolyzed by endogenous PDEs, predominantly PDE4 in the host cells such as HEK293, and is counted as a baseline. In the presence of a PDE4 inhibitor, cAMP accumulates and the increased levels of cAMP bind to and open the CNG cation channels, resulting in an influx of cations including Na⁺ and Ca²⁺. The cation influx triggers a cell membrane depolarization which can be quantified, for example, using a fluorescent membrane potential dye. This cell-based assay uses an artificial cell system leading to high false positives, requires significant manipulation of cells including cell engineering and dye loading, and relies on an indirect readout for PDE activity.

125. The disclosed methods are directed towards using label-free biosensor cellular assays for detecting and screen inhibitors for phosphodiesterases (PDEs), such as PDE4s.

126. Also disclosed are methods for screening inhibitors for endogenous PDEs without cell engineering. The disclosed methods relate to cell synchronization which results in robust detection of PDE inhibition-induced cellular response in real time using label-free biosensors.

127. The non-invasive detection with label-free biosensors not only enables the direct measurements of PDE activity in native cells, but also allows for studying the impact of PDE inhibition by compounds on other cell signaling pathways and processes, such as GPCR signaling.

128. Unlike other cell-based assays which require co-expression of a constitutively active Gₛ-coupled receptor as a driver for continued cAMP production and a CNG ion channel as a sensor to convert the PDE activity into a readout (i.e., cell membrane potential changes), the present disclosed methods do not require any cell engineering.

129. The disclosed methods also work for engineered cells, in which the cell engineering can boost the activity in living cells. This is unlike conventional cellular PDE4 assays which are often based on a single readout (i.e., a genetic reporter, a change in membrane potential, or a change in intracellular Ca²⁺ level). The present assays are also substantially simplified, and certain embodiments can be performed without washing. The use of cells engineered with a PDE could lead to boost the sensitivity of label-free cellular assays to detect the activity of the PDE engineered.

130. Furthermore, the disclosed methods do not require an addition of exogenous cAMP stimuli such as adenylate cyclase activator forskolin or GPCR agonist, because there are several endogenous Gₛ-coupled receptors that can provide a driving force for basal cAMP production. Instead, the present methods rely on cell synchronization to cause an alteration in cellular status, such as basal cAMP level in the cells, such that inhibition of a PDE (either endogenous or engineered) can lead to detectable cellular responses using the label-free biosensor.

### C. LABEL FREE CELL BASED ASSAYS

131. Label-free cell-based assays generally employ a biosensor to monitor compound-induced responses in living cells. The compound can be naturally occurring or synthetic, purified or unpurified mixture. A biosensor typically utilizes a transducer such as an optical, electrical, calorimetric, acoustic, magnetic, or like transducer, to convert a molecular recognition event or a ligand-induced change in cells contacted with the biosensor into a quantifiable signal. These label-free biosensors can be used for molecular interaction analysis, which involves characterizing how molecular complexes form and disassociate over time, or for cellular response, which involves characterizing how cells respond to stimulation. The biosensors that are applicable to the disclosed methods include, but not limited to, optical biosensor systems such as surface plasmon resonance (SPR) and resonant waveguide grating (RWG) biosensors including photonic crystal biosensor, resonant mirrors, or ellipsometer, and electric biosensor systems such as bioimpedance systems.

### D. BIOSENSORS

### 1. SPR and systems

132. Surface plasmon resonance (SPR) relies on a prism to direct a wedge of polarized light, covering a range of incident angles, into a planar glass substrate bearing an electrically conducting metallic film (e.g., gold) to excite surface plasmons. The resultant evanescent wave interacts with, and is absorbed by, free electron clouds in the gold layer, generating electron charge density waves (i.e., surface plasmons) and causing a reduction in the intensity of the reflected light. The resonance angle at which this intensity minimum occurs is a function of the refractive index of the solution close to the gold layer on the opposing face of the sensor surface. The compound addition is typically introduced by microfluidics, in conjunction with pumps and microchannels.

### 2. RWG Biosensors and Systems

133. A resonant waveguide grating (RWG) biosensor can include, for example, a substrate (e.g., glass), a waveguide thin film with an embedded grating structure, and a cell layer. The RWG biosensor utilizes the resonant coupling of light into a waveguide by means of a diffraction grating, leading to total internal reflection at the solution-surface interface, which in turn creates an electromagnetic field at the interface. This electromagnetic field is evanescent in nature, meaning that it decays exponentially from the sensor surface; the distance at which it decays to 1/e of its initial value is known as the penetration depth and is a function of the design of a particular RWG biosensor, but is typically on the order of about 200 nm. This type of biosensor exploits such evanescent waves to characterize ligand-induced alterations of a cell layer at or near the sensor surface.

134. RWG instruments can be subdivided into systems based on angle-shift or wavelength-shift measurements. In a wavelength-shift measurement, polarized light covering a range of incident wavelengths with a constant angle is used to illuminate the waveguide; light at specific wavelengths is coupled into and propagates along the waveguide. Alternatively, in angle-shift instruments, the sensor is illuminated with monochromatic light and the angle at which the light is resonantly coupled is measured. The resonance conditions are influenced by the cell layer (e.g., cell confluency, adhesion and status), which is in direct contact with the surface of the biosensor. When a ligand or an analyte interacts with a cellular target (e.g., a GPCR, a kinase) in living cells, any change in local refractive index within the cell layer can be detected as a shift in resonant angle (or wavelength).

135. The Corning® Epic® system uses RWG biosensors for label-free biochemical or cell-based assays (Corning Inc., Corning, N.Y.). The Epic® System consists of an RWG plate reader and SBS (Society for Biomolecular Screening) standard microtiter plates. The detector system in the plate reader exploits integrated fiber optics to measure the shift in wavelength of the incident light, as a result of ligand-induced changes in the cells. A series of illumination-detection heads are arranged in a linear fashion, so that reflection spectra are collected simultaneously from each well within a column of a 384-well microplate. The whole plate is scanned so that each sensor can be addressed multiple times, and each column is addressed in sequence. The wavelengths of the incident light are collected and used for analysis. A temperature-controlling unit can be included in the instrument to minimize spurious shifts in the incident wavelength due to the temperature fluctuations. The measured response represents an averaged response of a population of cells. The compound addition is introduced by either on-board pipettor or external liquid handler.

### 3. Electrical Biosensors and Systems

136. Electrical biosensors consist of a substrate (e.g., plastic), an electrode, and a cell layer. In this electrical detection method, cells are cultured on small gold electrodes arrayed onto a substrate, and the system's electrical impedance is followed with time. The impedance is a measure of changes in the electrical conductivity of the cell layer. Typically, a small constant voltage at a fixed frequency or varied frequencies is applied to the electrode or electrode array, and the electrical current through the circuit is monitored over time. The ligand-induced change in electrical current provides a measure of cell response. Impedance measurement for whole cell sensing was first realized in 1984. Since then, impedance-based measurements have been applied to study a wide range of cellular events, including cell adhesion and spreading, cell micromotion, cell morphological changes, and cell death. Classical impedance systems suffer from high assay variability due to use of a small detection electrode and a large reference electrode. To overcome this variability, the latest generation of systems, such as the CellKey system (MDS Sciex, South San Francisco, Calif.) and RT-CES (ACEA Biosciences Inc., San Diego, Calif.), utilize an integrated circuit having a microelectrode array. The compound addition is introduced by on-board pipettor.

### 4. High Spatial Resolution Biosensor Imaging Systems

137. Optical biosensor imaging systems, including SPR imaging system, ellipsometry imaging, and RWG imaging system, offer high spatial resolution, and are preferably used in the disclosed methods. For example, SPR Imager®II (GWC Technologies Inc) uses prism-coupled SPR, and takes SPR measurements at a fixed angle of incidence, and collects the reflected light with a CCD camera. Changes on the surface are recorded as reflectivity changes. Thus SPR imaging collects measurements for all elements of an array simultaneously.

138. Recently, Corning Incorporated also disclosed a swept wavelength optical interrogation system based on RWG biosensor for imaging-based application. In this system, a fast tunable laser source is used to illuminate a sensor or an array of RWG biosensors in a microplate format. The sensor spectrum can be constructed by detecting the optical power reflected from the sensor as a function of time as the laser wavelength scans, and analysis of the measured data with computerized resonant wavelength interrogation modeling results in the construction of spatially resolved images of biosensors having immobilized receptors or a cell layer. The use of image sensor naturally leads to an imaging based interrogation scheme. 2 dimensional label-free images can be obtained without moving parts.

139. Alternatively, Corning® Epic® angular interrogation system with transverse magnetic or p-polarized TM₀ mode can also be used. This system consists of a launch system for generating an array of light beams such that each illuminates a RWG sensor with a dimension of approximately 200 μm×3000 μm or 200 μm×2000 μm, and a CCD camera-based receive system for recording changes in the angles of the light beams reflected from these sensors. The arrayed light beams are obtained by means of a beam splitter in combination with diffractive optical lenses. This system allows up to 49 sensors (in a 7×7 well sensor array) to be simultaneously sampled at every 3 seconds.

140. Alternatively, a scanning wavelength interrogation system can also be used. In this system, a polarized light covering a range of incident wavelengths with a constant angle is used to illuminate and scan across a waveguide grating biosensor, and the reflected light at each location can be recorded simultaneously. Through scanning, a high resolution image across a biosensor can also be achieved. In all alternatives, the compound addition is introduced by either on-board pipettor or external liquid handler.

### E. LABEL-FREE BIOSENSOR CELLULAR ASSAYS MANIFEST LIGAND-INDUCED DYNAMIC MASS REDISTRIBUTION (DMR) SIGNALS IN LIVING CELL

141. Cell signaling mediated through a cellular target is encoded by spatial and temporal dynamics of downstream signaling networks. The coupling of temporal dynamics with spatial gradients of signaling activities guides cellular responses upon stimulation. Monitoring the integration of cell signaling in real time, if realized, would provide a new dimension for understanding cell biology and physiology. Optical biosensors including resonant waveguide grating (RWG) biosensor manifest a physiologically relevant and integrated cellular response related to dynamic redistribution of cellular matters, thus providing a non-invasive means for studying cell signaling.

142. Common to all optical biosensors is that they measure changes in local refractive index at or very near the sensor surface. Almost all optical biosensors are applicable for cell sensing in principle—they can employ an evanescent wave to characterize ligand-induced change in cells. The evanescent-wave is an electromagnetic field, created by the total internal reflection of light at a solution-surface interface, which typically extends a short distance (˜hundreds of nanometers) into the solution with a characteristic depth, termed as penetration depth or sensing volume.

143. Recently, theoretical and mathematical models were developed that describe the parameters and nature of optical signals measured in living cells in response to stimulation with ligands. These models, based on a 3-layer waveguide system in combination with known cellular biophysics, provide a link from ligand-induced optical signals to several cellular processes mediated through a receptor.

144. Given that the biosensor measures an averaged response of cells located at the area illuminated by the incident light, a cell layer of highly confluency is used in order to achieve optimal assay results. Because of the large dimension of cells compared to the short penetration depth of a biosensor, the sensor configuration is considered as a non-conventional three-layer system: a substrate, a waveguide film with a grating structure, and a cell layer. Thus, a ligand-induced change in effective refractive index (i.e., the detected signal) is, to first order, directly proportional to the change in refractive index of the bottom portion of cell layer:

ΔN=S(C)Δn_{c} (1)

where S(C) is the sensitivity to the cell layer, and Δn_{c} the ligand-induced change in local refractive index of the cell layer sensed by the biosensor. Because the refractive index of a given volume within a cell is largely determined by the concentrations of bio-molecules such as proteins, Δn_{c} can be assumed to be directly proportional to ligand-induced change in local concentrations of cellular targets or molecular assemblies within the sensing volume. Considering the exponentially decaying nature of the evanescent wave extending away from the sensor surface, the ligand-induced optical signal is governed by:

$$Δ N = S ( C ) α d {\sum }_{i} Δ {C}_{i} [ {e}^{\frac{- {z}_{i}}{Δ {Z}_{C}}} - {e}^{\frac{- {z}_{i + 1}}{Δ {Z}_{C}}} ] \qquad ( 2 )$$

where ΔZ_{c} is the penetration depth into the cell layer, α the specific refraction increment (about 0.18/mL/g for proteins), zᵢ the distance where the mass redistribution occurs, and d an imaginary thickness of a slice within the cell layer. Here the cell layer is divided into an equal-spaced slice in the vertical direction. Eq. 2 indicates that the ligand-induced optical signal is a sum of mass redistribution occurring at distinct distances away from the sensor surface, each with an unequal contribution to the overall response. Furthermore, the detected signal, in terms of wavelength or angular shifts, is primarily sensitive to mass redistribution occurring perpendicular to the sensor surface. Because of its dynamic nature, it is also referred to as dynamic mass redistribution (DMR) signal.

145. Cells rely on multiple cellular pathways or machineries to process, encode and integrate the information received. Unlike the affinity analysis with optical biosensors that specifically measures the binding of analytes to a protein target, living cells are much more complex and dynamic.

146. To study cell signaling, cells are brought to contact with the surface of a biosensor, which can be achieved through cell culture. These cultured cells are attached onto the biosensor surface through three types of contacts: focal contacts, close contacts and extracellular matrix contacts, each with its own characteristic separation distance from the surface. Depending on cell types as well as surface chemistry of the biosensor surface, cells could employ one or more of these types of contacts to become adherent on the biosensor surface. As a result, the basal cell membranes are generally distant away from the surface by ˜10-100 nm. These biosensors are able to sense the bottom portion of cells.

147. Cells, in many cases, exhibit surface-dependent adhesion and proliferation. In order to achieve robust cell assays, the biosensor surface could require a coating to enhance cell adhesion and proliferation. On the other hand, the surface properties could have direct impact on cell biology. For example, surface-bound ligands can influence the response of cells, while the mechanical compliance of a substrate material, which dictates how it will deform under forces applied by the cell, is influential. Together with the culture conditions (time, serum concentration, confluency, etc.), cellular status obtained can be distinct from one surface to another, and from one condition to another. Thus, special attentions to control cellular status are necessitated for developing biosensor-based cell assays.

148. Cells are dynamic objects with relatively large dimensions—typically tens of microns. Even without stimulation, cells constantly undergo micromotion—a dynamic movement and remodeling of cellular structure, as observed in tissue culture by time lapse microscopy at the sub-cellular resolution, as well as by bio-impedance measurements at the nanometer level.

149. Under un-stimulated conditions the cells generally give rise to an almost net-zero DMR response, as examined with RWG biosensor. This is partly because of the low spatial resolution of optical biosensors, as determined by the large size of the laser spot and the long propagation length of the coupled light. The size of the laser spot determines the size of the area studied—usually only one analysis point can be tracked at a time. Thus, the biosensor typically measures an averaged response of a large population of cells located at the light incident area. Although cells undergo micromotion at the single cell level, the large populations of cells examined give rise to a net-zero DMR response in average. Furthermore, it is known that intracellular macromolecules are highly organized and spatially restricted to appropriate sites in mammalian cells. The localization of proteins is tightly controlled in order for cells to regulate the specificity and efficiency of proteins interacting with their proper partners, to spatially separate protein activation and deactivation mechanisms, and thus to determine specific cell functions and responses. Thus, under un-stimulated conditions, the local mass density of cells within the sensing volume can reach an equilibrium state, thus leading to a net-zero optical response. It is worthy noting that the cells examined often have been cultured under conventional culture condition for a period of time such that most of the cells have just completed a single cycle of division.

150. Living cells have exquisite abilities to sense and respond to exogenous signals. Cell signaling was originally thought to function via linear routes where an environmental cue would trigger a linear chain of reactions resulting in a single well-defined response. However, amassing evidences show that cellular responses to external stimuli are much more complicated. It has become apparent that the information the cells received is processed and encoded into complex temporal and spatial patterns of phosphorylation and topological relocation of signaling proteins. The spatial and temporal targeting of proteins to appropriate sites is crucial to regulate the specificity and efficiency of protein-protein interactions, thus dictating the timing and intensity of cell signaling and responses. Pivotal cellular decisions, such as cytoskeletal reorganization, cell cycle checkpoints and apoptosis, depend on the precise temporal control and relative spatial distribution of activated signal-transducers. Thus, cell signaling mediated through a cellular target such as G protein-coupled receptor (GPCR) typically proceeds in an orderly and regulated manner, and consists of a series of spatial and temporal events, many of which lead to changes in local mass density or redistribution in local cellular matters of cells. These changes or redistribution when occurring within the sensing volume can be followed directly in real time using optical biosensors. As results, the resultant DMR signal is a novel physiological response of living cells, contains systems cell biology information of a ligand-receptor pair in living cells, and DMR signal contains systems cell pharmacology information of ligands acting on living cells.

### F. LABEL-FREE BIOSENSOR PDE CELLULAR ASSAYS

151. According to the present disclosed methods, robust detection of PDE activity in native cells under synchronization conditions is due to unique pre-compartmentalization of signaling complexes including PDE4. The inhibition of PDE4 activity in synchronized cells leads to a local increase of intracellular cAMP which could pass a threshold level required to activate downstream signaling events.

152. Specifically, disclosed herein are methods to synchronize the cellular status such that the inhibitory activity of any PDEs, particularly PDE4 by molecules can be robustly detected without need of cell engineering using label-free biosensor cellular assays. Without cellular synchronization the inhibitor induced PDE4 activity may also be detectable in living cells by biosensor cellular assays, but it likely leads to a right shift in potency of PDE inhibitors and/or less robust/reproducible assay results.

153. The disclosed methods do not require an addition of exogenous cAMP stimuli such as forskolin (an AC activator) or GPCR agonists. The disclosed methods are also non-invasive and label-free. This is unlike conventional PDE cellular assays, wherein certain labels (e.g., membrane potential dyes, or Ca2+-sensitive Fura-2 dye) or manipulations (e.g., cell engineering or voltage-clamping) are needed. In addition, for the disclosed methods, the assay protocol is substantially simplified, making this assay suitable for high-throughput screening.

154. The present methods are related to using label-free biosensor cellular assays for detecting PDE activity in native cells and screening PDE inhibitors using synchronized native cells. Also the disclosed methods can be used to study the PDE inhibition-induced cell signaling, and the impact of PDE inhibition on receptor biology such as GPCR signaling. The disclosed methods are not limited to native cells. The disclosed methods can also be applied to engineered cells such as stably or transitly transfected cells.

1. Cell Synchronization

155. The cell synchronization that renders PDE activity for being measured robustly can be achieved through at least three means, which can be used independently or in any combination of ways.

a) Ultra-High Confluency Culturing

156. In one method, the cells are synchronized through culturing. Here high initial seeding numbers of cells are used such that the cells reach high confluency early (i.e., the time being close to a single cycle of cell duplication), and undergo quiescence through continuous culture in either serum rich medium or in serum-free medium for an extended period of time (typically overnight). During such culture condition, cells reach ultra high confluency (>99%), and are in a fully quiescent state via the combination of contact inhibition and serum withdrawal.

b) Incubation Under Low CO₂ Environment

157. In another method, the cells are synchronized through incubation in a specific buffered solution. Here regular seeding numbers of cells can be used to culture cells onto the sensor surface until it reach high confluency (>90%). Afterwards, the cells are washed and maintained in an assay buffered solution at physiological pH (e.g., pH 7.1). After incubation under a low (<4.5%) CO2 environment for an extended period of time (typically longer than 90 min, such as 2 hrs, 3 hrs, 4 hrs, 5 hrs, 6 hrs, 8 hrs, etc), the cells are then assayed.

c) Pretreatment with a Carbonic Anhydrase Inhibitor

158. In one method, the cells are synchronized through pretreatment with a carbonic anahydrase inhibitor before the assay. After culture and washing, the cells are incubated in an assay solution with or without a pH controlling agent but containing the carbonic anahydrase inhibitor. After further incubation in the detector system, the cells are assayed.

159. Disclosed are methods to screen PDE4 inhibitors comprising: Providing a biosensor wherein the biosensor is preferably located within a well of microtiter plate; Seeding cells onto the said biosensor surface wherein the seeding numbers of cells are high enough to achieve high confluency early time during culture; Culturing cells under serum containing medium an extended period of time; Optionally starving the cells for another period of time until the cells reach high confluency and quiescent state; Washing the quiescent cells with a pH-buffered assay solution; Maintaining the quiescent cells with the said pH-buffered assay solution in a biosensor detector system; and assaying the cellular response triggered by compounds, wherein a compound-induced biosensor signal similar to those Gs coupled receptor in the same cellular background is an indicator of PDE4 inhibition.

160. Also disclosed are methods to screen PDE4 inhibitors, comprising: Providing a biosensor wherein the biosensor is preferably located within a well of microtiter plate; Seeding cells onto the said biosensor surface wherein the seeding numbers of cells are high enough to achieve high confluency after culture; Capturing cells under serum rich medium; Optionally starving the cells for another period of time with serum depleted medium; Washing the cells with a pH buffered assay solution; Maintaining the cells with the said assay solution in a biosensor detector system under low CO2 environment for at least 2 hours; and assaying the cellular response triggered by compounds, wherein a compound-induced biosensor signal similar to those Gs coupled receptors in the same cellular background is an indicator of PDE4 inhibition.

161. Also disclosed are methods to screen PDE4 inhibitors comprising: Providing a biosensor wherein the biosensor is preferably located within a well of microtiter plate; Seeding cells onto the said biosensor surface wherein the seeding numbers of cells are high enough to achieve high confluency after culture; Culturing cells under serum rich medium; Optionally starving the cells for another period of time with serum depleted medium; Washing the cells with a pH buffered assay solution; Maintaining and incubate the cells with the said assay solution in the presence of a carbonic anahydrase inhibitor in a biosensor detector system for at least 2 hours; and assaying the cellular response triggered by compounds, wherein a compound-induced biosensor signal similar to those Gs coupled receptors in the same cellular background is an indicator of PDE4 inhibition.

162. Also disclosed are methods of incubating cells on a biosensor, comprising the steps of: a. providing a biosensor, b. seeding cells onto the biosensor surface; c. culturing cells under serum containing medium; and d. synchronizing the cells.

163. Also disclosed are methods, wherein the step of synchronizing further comprises the steps of treating cells to reach high confluency and quiescent state, washing the cells with a pH-buffered assay solution, maintaining the cells with the pH-buffered assay solution in a biosensor detector system, wherein the step of synchronizing further comprising the steps of: starving the cells for another period of time with serum depleted medium and washing the cells with a pH buffered assay solution, maintaining the cells with the assay solution in a biosensor detector system under low CO2 environment for another period of time, and/or wherein the step of synchronizing further comprising the steps of: starving the cells for another period of time with serum depleted medium; washing the cells with a pH buffered assay solution; maintaining and incubating the cells with the assay solution with a carbonic anahydrase inhibitor in the biosensor detector system for another period of time.

164. Also disclosed are methods, further comprising the steps of contacting the cell with a test compound wherein the contact produces a biosensor response from the cell, and using the biosensor response to determine if the test compound is a PDE modulator.

165. Disclosed are methods wherein the biosensor is located within a well of microtiter plate, wherein the number of seeding cells achieve high confluency early during culture, wherein the number of seeding cells achieve high confluency after culture, wherein the cells is culturing for an extended period of time, wherein the cells reach high confluency and quiescent state by being starved, wherein PDE inhibition is indicated if the test compound-induced biosensor signal is similar to a receptor's signal in the same cellular background, wherein the PDE is PDE4, wherein the compound-induced biosensor signal similar to the Gs coupled receptors in the same cellular background indicates that the compound inhibits a PDE4, wherein greater than 90% of the cells have undergone only one cell division, wherein the cells comprise at least 90% native cells, wherein the cells are grown to ultra high confluency, wherein the biosensor is located within a well of microtiter plate, wherein the cells are maintained with the assay solution in a biosensor detector system under low CO2 environment for 30 min, 1 hr, 2 hrs, 3 hrs, 4 hrs, 5 hrs, 6 hrs, 8 hrs, 10 hrs, 15 hrs, 20 hrs, 30 hrs, 40 hrs, 50 hrs, or 100 hrs, wherein the cells are maintained with the assay solution in the biosensor detector system under low CO2 environment for at least 2 hrs, wherein the concentration of the CO2 environment in the biosensor detection system is below 3.5%, wherein the cells are maintained and incubated with the assay solution with a carbonic anahydrase inhibitor in the biosensor detector system for 30 min, 1 hr, 2 hrs, 3 hrs, 4 hrs, 5 hrs, 6 hrs, 8 hrs, 10 hrs, 15 hrs, 20 hrs, 30 hrs, 40 hrs, 50 hrs, or 100 hrs, wherein the cells are maintained and incubated with the assay solution with a carbonic anahydrase inhibitor in the biosensor detector system for at least 2 hrs, wherein step d. is omitted, wherein the cells are synchronized via culturing and wherein the cells are cultured for an extended period of time, and/or wherein the cells reach high confluency and quiescent state by being starved.

166. Any combinations of the above synchronization methods can be used for PDE inhibitors screening.

### VI. EXAMPLES

### a) Material and Methods

(1) Materials

167. IBMX, IC63197, Ro-20-1724, R-rolipram, YM-976, siguazodan, cilostazol, cilostamide, milrinone, anagrelide, MY05445, zaprinast, BRL50481, ibudilast, and MMPX were purchased from Tocris Chemical Co. (St. Louis, Mo.). Acetazolamide, histamine, epinephrine, nicotinic acid, pinacidil, poly (I:C), forskolin, and epidermal growth factor (EGF) were obtained from Sigma Chemical Co. (St. Louis, Mo.). Tyrphostins 25, tryphostin 51 and Phorbol 12-myristate 13-acetate (PMA) were obtained from BioMol International Inc (Plymouth Meeting, Pa.). SLIGKV-amide were obtained from BaChem Americas Inc. (Torrance, Calif.). Epic® 384 biosensor microplates were obtained from Corning Inc. (Corning, N.Y.).

(2) Cell Culture

168. All cell lines were obtained from American Type Cell Culture (Manassas, Va.). The cell culture medium used was: Dulbecco's modified Eagle's medium (DMEM) supplemented with 10% fetal bovine serum (FBS), 4.5 g/liter glucose, 2 mM glutamine, and antibiotics for human epidermoid carcinoma A431 and human lung carcinoma A549.

169. Cells were typically grown using ˜1 to 2×10⁴ cells per well at passage 3 to 15 suspended in 50 μl of the corresponding culture medium in the biosensor microplate, and were cultured at 37° C. under air/5% CO₂ for ˜1 day. Except for A431 which was subject to ˜20 hr starvation through continuously culture in the serum-free DMEM, the other cell types were directly assayed without starvation. The confluency for all cells at the time of assays was ˜95% to 100%.

(3) RNA Isolation

170. RNA was isolated from the A431 and A549 cell cultures. Total RNA was extracted with the use of RNeasy® mini kit (Qiagen). Residual DNA was removed by ribonuclease-free deoxyribonuclease (DNase) I treatment (RNase-Free DNase set from Qiagen).

(4) RT-PCR

171. RNAs were reversely transcribed using 1 μg of total RNA in the presence of SuperScript III One-step RT-PCR system with Platinum® Taq High Fidelity (Invitrogen). The cycling conditions were the following:

172. cDNA synthesis: 30 min at 50° C. followed by 5 min denaturation at 95° C.

173. PCR amplification: 35 cycles of 95° C. for 30 sec, 50° C. for 30 sec and 72° C. for 2 min. This was followed by a final extension of 10 min at 65° C.

174. Primers for PDE3A and PDE3B were used as described previously (F. Murray et al, British Journal of Pharmacology (2002) 137, 1187±1194). The PDE3A sense primer was CTG GCC AAC CTT CAG GAA TC and the antisense primer was GCC TCT TGG TTT CCC TTT CTC. The PDE3B sense primer was AAT CTT GGT CTG CCC ATC AGT CC and the antisense primer was TTC AGT GAG GTG GTG CAT TAG CTG. In each PCR reaction, 100 ng of primers of GAPDH, as internal control were added. The GAPDH primer sequences were GACCCCTTCATTGACCTCAACTACATG39 (sense) and GTCCACCACCCTGTTGCTGTAGCC39 (antisense).

175. The PCR products were analyzed by electrophoresis on 3% agarose gel. The identity of PCR products was confirmed by comparing the size of the product with the size expected from the gene sequence.

(5) Chemiluminescence Quantification of Cyclic Amp

176. A431 cells were seeded in 96 well TCT microplate at 80000 cells/well, cultured for 20 hrs and starved for an extra 20 hrs. Cells were washed two time with HBSS and incubated with 100 μl of HBSS, Leibovitz's L-15 medium (Gibco) or 1 μM of Acetozamine (Sigma) in HBSS for 2 hrs in the Epic instrument. Compounds (epinephrine, IBMX or HBSS) were then added to the plate and cells were exposed for 15 mins at room temperature. The medium or buffer was removed and cells were lysed in 100 μm al of lysis buffer (from cAMP HTS immunoassay kit, Millipore) containing 1 mM IBMX. 50 μl of cell extracts as well as cAMP standard were then analyzed using the same procedure described by the vendor.

(6) Optical Biosensor System and Cell Assays

177. Epic® wavelength interrogation system (Corning Inc., Corning, N.Y.) was used for whole cell sensing. This system consists of a temperature-control unit, an optical detection unit, and an on-board liquid handling unit with robotics. The detection unit is centered on integrated fiber optics, and enables kinetic measures of cellular responses with a time interval of ˜15 sec. The compound solutions were introduced by using the on-board liquid handling unit (i.e., pippetting).

178. The RWG biosensor is capable of detecting minute changes in local index of refraction near the sensor surface. Since the local index of refraction within a cell is a function of density and its distribution of biomass (e.g., proteins, molecular complexes), the biosensor exploits its evanescent wave to non-invasively detect ligand-induced dynamic mass redistribution in native cells. The evanescent wave extends into the cells and exponentially decays over distance, leading to a characteristic sensing volume of ˜150 nm, implying that any optical response mediated through the receptor activation only represents an average over the portion of the cell that the evanescent wave is sampling. The aggregation of many cellular events downstream the receptor activation determines the kinetics and amplitudes of a ligand-induced DMR.

179. For biosensor cellular assays, compound solutions were made by diluting the stored concentrated solutions with the HBSS (1× Hanks balanced salt solution, plus 20 mM Hepes, pH 7.1), and transferred into a 384 well polypropylene compound storage plate to prepare a compound source plate. Two compound source plates were made separately when a two-step assay was performed. In parallel, the cells were washed twice with the HBSS and maintained in 30 μl of the HBSS to prepare a cell assay plate. Both the cell assay plate and the compound source plate(s) were then incubated in the hotel of the reader system. After incubation the baseline wavelengths of all biosensors in the cell assay microplate were recorded and normalized to zero. Afterwards, a 2 to 10 min continuous recording was carried out to establish a baseline, and to ensure that the cells reached a steady state. Cellular responses were then triggered by transferring 10 μl of the compound solutions into the cell assay plate using the on-board liquid handler.

180. All studies were carried out at a controlled temperature (28° C.). At least two independent sets of experiments, each with at least three replicates, were performed. The assay coefficient of variation was found to be <10%.

### b) Example 1

### Expression Patterns of Endogenous Phosphodiesterases in A431 Cells

181. PDE4 is predominantly expressed in inflammatory cells, including eosinophils, neutrophils, T lymphocytes, macrophages, and mast cells. Abnormal levels of PDE4 isoenzymes are considered to underlie some of the immune and inflammatory abnormalities of atopic diseases. Previously RT-PCR and immunoblotting were used to study the expression of PDE4 isoforms in A431 cells. Results showed that A431 endogenously expresses all PDE4 isoforms, and PDE4D is the most abundant PDE4 isoform in A431 cells (Chujor, C. S. N., Hammerschmid, F., and Lam, C. Cyclic nucleotide phosphodiesterase 4 subtypes are differentially expressed by primary keratinocytes and human epidermoid cell lines. J. Investigative Dermatology, 1998, 110, 287-291).

182. Here we were focused on the expression analysis of PDE3 isoforms in A431. Results, as shown in FIG. 1, revealed that A431 only expresses low level of PDE3A, but almost no PDE3B isoform.

### c) Example 2

### Basal Intracellular cAMP level of A431 Cells Under Different Synchronized Conditions

183. Cyclic AMP (cAMP) is a diffusible intracellular second messenger that is produced in response to hormone action. The release of cAMP into the cytoplasm is initiated by the occupancy of GPCRs at the plasma membrane by several different ligands, including adrenocorticotropin, glucagon and adrenaline. The ligand-bound GPCR catalyzes the exchange of GDP for GTP on the α-subunit of the associated heterotrimeric G protein, resulting in the activation of the α-subunit and its dissociation from the βγ-dimer. Both the α- and βγ-subunits can then initiate or inhibit distinct intracellular signaling cascades. The α-subunit of the Gs subtype activates adenylyl cyclase, which converts ATP to cAMP. GPCR-mediated downstream signaling is terminated by the intrinsic GTPase activity of the α-subunit, which hydrolyses GTP to GDP. The net effect is the intracellular generation of cAMP at points that emanate from the plasma membrane. cAMP was initially considered to be a second messenger that diffused freely throughout the cell with a theoretical range-of-action of 220 μm. However, advances in live-cell imaging have visualized gradients, rather than a uniform intracellular distribution, of cAMP, which indicates that this second messenger accumulates at specific sites within cells. Several proteins, such as cyclic nucleotide-gated channels, phosphodiesterases, and guanine nucleotide-exchange proteins activated by cAMP (EPACs), bind to and are activated by cAMP. The localized activation of such cAMP-binding proteins would therefore enable this ubiquitous second messenger to be used to propagate diverse responses.

184. It is widely believed that without significant cell engineering, it is almost impossible to assay the activity of endogenous PDEs, including PDE4 isoforms, in native cells. We here speculated that synchronization of cells under certain conditions would not only result in an appropriate basal level of intracellular cAMP, but also lead to an appropriate pre-organization of these signaling molecules, such that the inhibitory effect of PDE activity by PDE inhibitors would lead to a detectable signal using non-invasive and label free biosensors. To test this hypothesis, we first examined the basal level of intracellular cAMP under different synchronized conditions.

185. As shown in FIG. 2, A431 cells were used as a model. A431 Cells were cultured in a serum rich medium on Corning tissue culture treated 96 well microplate until it reached a high confluency (˜95%). After washing, the cells were continuously cultured in a serum free medium for overnight. At this point, the cells were at ˜100% confluency. After washing with corresponding solution, the cells are incubated at the same low CO₂ environment (˜2% CO₂) for 2 hr. The cells were then lysed with a cell lysis buffer containing 100 micromolar IBMX to block the PDE activity, followed by cAMP measurements, using the protocol as recommended by the supplier. Results showed that the 2 hr synchronization of the quiescent A431 cells in the HBSS (pH 7.1) resulted in a moderate cAMP level (˜1.0±0.1 pmole/160k cells), while the 2 hr synchronization of the quiescent cells in the CO₂ independent L-15 medium led to the highest basal cAMP level (1.16±0.1 μmole/160K cells), and the quiescent cells synchronized in the L-15 medium containing 1 micromolar carbonic anahydrase inhibitor acetazolamide resulted in the lowest basal cAMP level (0.74±0.1 pmole/160K cells). Consistent with our speculation is that the basal cAMP level of a cell can be controlled through synchronization. Data also showed that IBMX at concentration range between 10 nanomolar to 100 micromolar had little impact on the intracellular cAMP levels under all conditions, as measured at such whole cell lysate level (data not shown).

### d) Example 3

### The Non-Selective PDE Inhibitor IBMX LED to a Robust Gs-DMR Signal in Quiescent A431 Cells Under Certain Synchronized Conditions

186. The results in FIG. 2 confirmed our hypothesis that the cell synchronization can be used to achieve desired intracellular cAMP level. At the whole cell level, it is obvious that it is difficult to measure endogenous PDE activity, as showed in the cAMP measurement. However, we hypothesized that the threshold of increased intracellular cAMP level upon stimulation to trigger cell signaling is much lower than those reported in literature using either cAMP fluorescence biosensor method or cell lysate whole cell measurements, and therefore the inhibition of endogenous PDEs in cells obtained under certain synchronization conditions would be able to raise the localized intracellular cAMP level high enough to trigger cell signaling, thus rendering the label-free biosensor measurements of PDE activity possible. To test this hypothesis, we first examined the relative potency of the endogenous Gₛ-coupled β2-adrenergic receptor agonist epinephrine to trigger DMR signal and whole cell cAMP accumulation. Results showed that epinephrine exhibited much higher potency to activate the DMR signal obtained using the label-free Epic® system than that to cause cAMP accumulation measured using the whole cell cAMP kit. This result indicates that the relative low occupancy of the Gₛ-coupled β2-adrenergic receptor by epinephrine is sufficient to trigger the DMR signal, and the threshold of increased, possibly initially localized, cAMP is much lower than expected to fully activate the cell signaling including the DMR signal.

187. We further examined the DMR signal, if any, of the quiescent A431 cells upon treatment with PDE inhibitors. In agreement with our hypothesis, we found that label-free cellular PDE assays are feasible, but are sensitive to cell synchronization conditions. The main results were summarized in FIG. 4. The quiescent A431 cells obtained under the same culture condition were used. Here the initial seeding numbers of cells were relatively low (˜20K cells per well in a 384 well Epic biosensor microplate tissue culture treated). After cultured in the serum rich medium for ˜24 hrs, the cells just reach a confluency of ˜95%. After washing with the serum free medium, the cells were continuously cultured in the serum free medium for another 24 hrs to reach a quiescent state. Here the quiescent state was largely achieved through serum withdrawl. The quiescent A431 cells were further synchronized in the CO₂ independent medium led to little DMR signals even at the highest doses of the non-selective PDE inhibitor IBMX examined (FIG. 4c). Under this synchronized condition, the basal cAMP level is high (FIG. 2). However, the quiescent A431 cells obtained under the same culture condition, but synchronized in the CO₂ independent L-15 medium containing the carbonic anahydrase inhibitor acetamolamide at 1 micromolar, led to a robust DMR signal which displayed classical dose dependency (FIG. 4D). Similarly, the quiescent A431 cells synchronized in the pH controlled assay buffer solution (i.e., 1×HBSS containing 20 mM Hepes, pH 7.1) with or without acetamolamide at low CO₂ environments for 2 hr responded to IBMX with a dose-dependent and robust DMR signal (FIG. 4A and FIG. 4B, respectively). We also showed that the same quiescent cells but synchronized in the same HBSS buffer solution for a shorter time (<90 min) led to an inconsistent DMR signal upon stimulation with IBMX or the PDE4 selective inhibitor rolipram (data not shown). These results indicate that synchronization of cells into a state having relatively lower basal level of intracellular cAMP is preferably useful for robust detection of endogenous PDE activity, therefore for screening PDE inhibitors. This is possibly that during the synchronization step, these quiescent cells tend to rebalance their intracellular cAMP concentration in response to environmental changes (e.g., low CO₂ concentration in the detector system). The cell membrane bound carbonic anahydrase is a known sensor for CO₂ level. Interestingly, under the three synchronized conditions tested wherein IBMX led to a detectable DMR signal, the potency of IBMX was found to be similar (˜EC50 of 10 micromolar). Also interesting is that the IBMX induced DMR signal is closely resembled to the classical Gₛ-type DMR signal in the same A431 cells (Fang, Y., and Ferrie, A. M. (2008) “label-free optical biosensor for ligand-directed functional selectivity acting on β₂ adrenoceptor in living cells”. FEBS Lett. 582, 558-564.).

188. In FIG. 4 the quiescent cells were achieved primarily through serum withdrawl. We were interested in the quiescent cells achieved through both contact inhibition and serum withdrawl. To do so, much higher initial seeding numbers of cells (>=25k cells per well for the 384 well Epic biosensor microplate) were used. After one day culture in the serum rich medium, the cells already reached compact monolayers, and underwent contact inhibition induced quiescence. After continuous culturing in the serum free medium for another day, the cells reached a different quiescent state. Compared to those in quiescent cells achieved through serum withdrawl only, the PDE4 inhibitor R-rolipram exhibited a much higher potency with an EC50 of 250 nM in the quiescent cells achieved through both contact inhibition and serum withdrawl (FIG. 5). Here both quiescent cells were subject to the same synchronization condition (2 hr incubation in the HBSS assay solution in a low CO₂ environment). Similar trend was also obtained for the non-selective PDE inhibitor IBMX (data not shown)

189. Since A431 cells predominately express PDE4 isoforms, and also express PDE3 isoforms but at relatively lower levels, we were interested in the selectivity of PDE inhibitors that are capable of triggering DMR signals. Here the quiescent A431 cells were used using 22k cells per well in 384 well Epic biosensor microplates, followed by one day (˜24 hrs) culture in serum medium and 1 day (˜24 hrs) serum free medium, and sequential synchronization in the HBSS buffer solution under a relatively low CO₂ environment for 2 hrs. Afterwards, the quiescent cells were subject to stimulation individually with a panel of PDE inhibitors, each at 12.5 micromolar. The main results were summarized in FIG. 6 to FIG. 11. For comparison, the DMR signal of cells stimulated with the buffer only (“Vehicle”) were included. As expected, the quiescent A431 cells responded to the buffer with a steady net-zero DMR signal. In contrast, all PDE4 selective inhibitors, including ICI63197, Ro-20-1724, R-rolipram and YM-976 led to a Gₛ-like DMR signal (FIG. 6). The difference in efficacy at the dose examined can reflect the difference in their relative potency and/or true efficacy due to their difference in isoform selectivity. Similarly, the PDE inhibitors that also led to a similar Gₛ-DMR signal in quiescent A431 cells included the two non-selective PDE inhibitors IBMX and tyrphostins 25 (FIG. 7), and the three potent but less selective PDE3 inhibitors siguazodan, cilostazol, and cilostamide (FIG. 8). Conversely, the two highly selective PDE3 inhibitors milrinone and anagrelide did not result in any detectable DMR signals (FIG. 9), the same was found for the three PDES selective inhibitors MY-5445, zaprinast and ibudilast (FIG. 10), the PDE7 selective inhibitor BRL-50481 and the PDE1 selective inhibitor MMPX (FIG. 11). Taken together, these results indicate that the Gₛ-DMR signals induced by various PDE inhibitors are due to their inhibitory effect on endogenous PDE4 isoforms. This reflects the unique expression patterns of endogenous PDE isoforms in A431 cells.

190. Although we used A431 cells as a model, the disclosed methods are also applicable to other types. Similar Gₛ-type DMR signals were obtained for the non-selective PDE inhibitors IBMX and tyrphostin 25 in human lung carcinoma A549 cells as well as human colon carcinoma HT-29 cells (data not shown). Interestingly, although it is beneficial, the serum withdrawl-induced quiescence is not necessary for measuring PDE4 activity in native A549 or HT-29 cells. In addition, although the Gₛ-like DMR signals induced by several PDE inhibitors were primarily attributed to PDE4 isoforms-induced cell signaling in A431, the present methods should be also applicable to other types of cAMP-related PDE isoforms, given that their expressions in a cell are high enough to trigger cell signaling.

### E) Example 4

### DMR Indexing Exhibited Polypharmacology of Tyrphostin 51

191. To characterize the polypharmacology including PDE4 inhibitory activity of compounds, we used the known EGFR inhibitor tyrphostin 51 as an example. Quiescent A431 and proliferating A549 cells were separately exposed to tyrphostin 51 at 10 micromolar for about 1 hr to generate its primary profiles, followed by stimulation with each marker at a fixed concentration for another 1 hr or so to generate secondary profiles of the tyrphostin 51 against the panels of markers in both types of cells. Next, one or more specific DMR parameters were chosen as a readout for plotting the DMR index of the molecule.

192. The first panel of markers was contacted with quiescent A431 cells at concentrations close to each of the marker's EC₈₀ or EC₁₀₀, and DMR measurements were obtained with and EPIC® system. This panel of markers was selected from a library of markers identified in A431 cells using Epic® cellular assays. This panel included epidermal growth factor (EGF), epinephrine, nicotinic acid, and histamine. Each of these markers leads to a wide array of cell signaling. Using Epic® cellular assays in conjunction with chemical biology and cell biology approach similar to the EGFR DMR mentioned above, we have determined the pathway(s) and network interaction(s) that is accounted for each marker-induced DMR signal in A431 cells. The main observations are summarized below. Epidermal growth factor is a natural agonist for endogenous EGF receptor in A431 whose activation leads to a divergent array of cell signaling, including PLC-PI3K pathway, MAPK pathway, STAT pathway, and PKC pathway. Epinephrine is a natural agonist for endogenous beat2-adrenergic receptor in A431 cells, whose activation leads to cAMP-PKA pathway. Nicotinic acid is a natural agonist for endogenous GPR109A receptor in A431, whose activation leads to Gi-mediated signaling and MAPK pathway. Histamine is a natural agonist for endogenous histamine type 1 receptor (H1R) in A431, whose activation leads to Gq-mediated signaling and PKC pathway. FIG. 7 shows the DMR signals of fully quiescent A431 cells in response to stimulation with the first panel of markers as determined by the Epic® cellular assays. Each DMR signal represents an averaged response of 4 replicates.

193. The second panel of markers was contacted with A549 cells at concentrations close to each of the marker's EC₈₀ or EC₁₀₀, and DMR measurements were obtained with and EPIC® system. This panel included poly(I:C), SLIGKV-amide, pinacidil, PMA, histamine, and forskolin. Each of these markers also leads to a wide array of cell signaling. Using Epic® cellular assays in conjunction with chemical biology and cell biology approach similar to the EGFR DMR mentioned above, we have determined the pathway(s) and network interaction(s) that is accounted for each marker-induced DMR signal in A549 cells. The main observations are summarized below. Poly(I:C) is an agonist for endogenous Toll-like receptor(s) in A549, whose activation leads to IKK pathway and AKT pathway. SLIGKV-amide is an agonist for endogenous protease activated receptor subtype 2 (PAR2) in A549, whose activation leads to G_{q}-, Gᵢ- and G_{12/13}-mediated signaling. Pinacidil is an opener for endogenous ATP-sensitive potassium (K_{ATP}) ion channel in A549, whose activation leads to Rho- and JAK-mediated signaling. PMA is an activator for protein kinase C(PKC), whose activation leads to PKC pathway and degranulation. Histamine is a natural agonist for endogenous histamine receptors (dominantly H1R, and possibly H3R), whose activation leads to dual signaling, possibly through G_{q} and Gᵢ mediated signaling in A549. Forskolin is an activator for adenylyl cyclases, whose activation leads to cAMP-PKA pathway in A549. FIG. 8 shows the DMR signals of A549 cells in response to stimulation with the second panel of markers as determined by the Epic® cellular assays. Each DMR signal represents an averaged response of 4 replicates.

194. For the pinacidil DMR response in A549, the amplitude of the N-DMR (i.e., the amplitude 30 min after stimulation) was chosen. For the poly(I:C) DMR response in A549, the amplitude of the late DMR (i.e., the amplitude 50 min after stimulation) was chosen. For the PMA response in A549, the late DMR amplitude (i.e., the amplitude 50 min after stimulation) was chosen. For the SLIGKV-amide DMR response in A549, the P-DMR amplitude (i.e., the amplitude 20 min after stimulation) was chosen. For the forskolin response in A549 cells, the P-DMR amplitude (i.e., the amplitude 50 min after stimulation) was chosen. For the histamine response in A549, both the early DMR amplitude (i.e., amplitude 10 min after stimulation) and the late response (i.e., the amplitude 30 min after stimulation) were chosen. For the epinephrine response in A431, the P-DMR amplitude (i.e., the amplitude 50 min after stimulation) was chosen. For the nicotinic acid DMR in A431, the P-DMR amplitude (i.e., the amplitude 3 min after stimulation) was chosen. For the EGF DMR in A431, both the P-DMR (i.e., the amplitude 4 min after stimulation) and N-DMR amplitudes (i.e., the decaying amplitude between 4 min and 40 min after simulation) were chosen. For the histamine response in A431, the P-DMR amplitude (i.e., the amplitude 3 min after stimulation) was chosen. The percentage of modulation of the marker against each marker was calculated by normalizing the secondary profile of the molecule against the marker in the cell to the primary profile of the same marker acting on the same cell.

195. As shown in FIG. 12, the DMR index of tyrphostin 51 can include two types of responses: the primary response profile (i.e., the tyrphostin 51-triggered primary profile) in both cell lines, and the antagonist mode DMR index (i.e., the secondary profiles of the ligand against the two panels of markers, each for one cell line).

196. FIGS. 12A and B show the primary profiles of tyrphostin 51 in A431 and A549 cells, respectively. The tyrphostin 51 DMR signals in both cell lines closely resembled the classic Gₛ-DMR signals, indicative of the activation of Gₛ pathway. Here four replicates of the tyrphostin 51 primary profiles in both cell lines were given to highlight the reproducibility of the assays. Here the ultra-high confluent (˜100%) cells were synchronized using the 2 hrs incubation in a low CO₂ (˜2%) environment. The modulation index of tyrphostin 51, as shown in FIG. 12C, exhibited that the pretreatment of cells with tyrphostin 51 partially attenuated the histamine DMR and epinephrine DMR signals in A431 cells, as well as the forskolin DMR signal in A549 cells, an indicator for the activation of Gₛ pathway. At the same time, the pretreatment of cells with tyrphostin 51 also caused attenuation of the EGF induced DMR signal in A431, an indicator for the inhibition of EGFR pathway. Taken together, these results show that tyrphostin 51 gave rise to polypharmacology, including EGFR and PDE4 inhibitory activities.

### B. REFERENCES

1. Fang, Y., Ferrie, A. M., Fontaine, N. H., and Yuen, P. K. (2005) “Characteristics of dynamic mass redistribution of EGF receptor signaling in living cells measured with label free optical biosensors”. Anal. Chem., 77, 5720-5725.
2. Fang, Y., Ferrie, A. M., Fontaine, N. H., Mauro, J. and Balakrishnan, J. (2006) “Resonant waveguide grating biosensor for living cell sensing”. Biophys. J., 91, 1925-1940.
3. Fang, Y., and Ferrie, A. M. (2008) “label-free optical biosensor for ligand-directed functional selectivity acting on β2 adrenoceptor in living cells”. FEBS Lett. 582, 558-564.
4. Fang, Y., Frutos, A. G., Verklereen, R. (2008) Label-free cell assays for GPCR screening. Comb. Chem.& HTS 11, 357-369.
5. Chujor, C. S. N., Hammerschmid, F., and Lam, C. Cyclic nucleotide phosphodiesterase 4 subtypes are differentially expressed by primary keratinocytes and human epidermoid cell lines. J. Investigative Dermatology, 1998, 110, 287-291
6. Bora, R. S., et al., A reporter gene assay for screening of PDE4 subtype selective inhibitors. Biochemical and Biophysical Research Communications, 2007, 356, 153-158;
7. Bora, R. S., et al., Development of a cell-based assay for screening of phosphodiesterase 10A (PDE10A) inhibitors using a stable recombinant HEK-293 cell line expressing high levels of PDE10A. Biotechnology and Applied Biochemistry, 2008, 49, 129-134.
Titus, S. A., et al., A cell-based PDE4 assay in 1536-well plate format for high-throughput screening. Journal of Biomolecular Screening 2008, 13, 609-618.

1. A method of incubating cells on a biosensor, comprising the steps of:
a. providing a biosensor;
b. seeding cells onto the biosensor surface;
c. culturing cells under serum containing medium, and
d. synchronizing the cells.

2. The method of claim 1, wherein the step of synchronizing further comprises the steps of treating cells to reach high confluency and quiescent state, washing the cells with a pH-buffered assay solution, maintaining the cells with the pH-buffered assay solution in a biosensor detector system.

3. The method of claim 1, wherein the step of synchronizing further comprising the steps of: starving the cells for another period of time with serum depleted medium and washing the cells with a pH buffered assay solution, maintaining the cells with the assay solution in a biosensor detector system under low CO₂ environment for another period of time.

4. The method of claim 1, wherein the step of synchronizing further comprising the steps of: starving the cells for another period of time with serum depleted medium; washing the cells with a pH buffered assay solution; maintaining and incubating the cells with the assay solution with a carbonic anahydrase inhibitor in the biosensor detector system for another period of time.

5. The method of claims 4, further comprising the steps of contacting the cell with a test compound wherein the contact produces a biosensor response from the cell, and using the biosensor response to determine if the test compound is a PDE modulator.

6. The method of claim 5, wherein the biosensor is located within a well of microtiter plate.

7. The method of claim 6, wherein the number of seeding cells achieve high confluency early during culture.

8. The method of claim 6, wherein the number of seeding cells achieve high confluency after culture.

9. The method of claim 6, wherein the cells is culturing for an extended period of time.

10. The method of claim 6, wherein the cells reach high confluency and quiescent state by being starved.

11. The method of claim 6, wherein PDE inhibition is indicated if the test compound-induced biosensor signal is similar to a receptor's signal in the same cellular background.

12. The method of claim 11, wherein the PDE is PDE4.

13. The method of claim 6, wherein the compound-induced biosensor signal similar to the Gs coupled receptors in the same cellular background indicates that the compound inhibits a PDE4.

14. The method of claim 6, wherein greater than 90% of the cells have undergone only one cell division.

15. The method of claim 6, wherein the cells comprise at least 90% native cells.

16. The method of claim 6, wherein the cells are grown to ultra high confluency.

17. The method of claim 6, wherein the biosensor is located within a well of microtiter plate.

18. The method of claim 6, wherein the cells are maintained with the assay solution in a biosensor detector system under low CO₂ environment for 30 min, 1 hr, 2 hrs, 3 hrs, 4 hrs, 5 hrs, 6 hrs, 8 hrs, 10 hrs, 15 hrs, 20 hrs, 30 hrs, 40 hrs, 50 hrs, or 100 hrs.

19. The method of claim 18, wherein the cells are maintained with the assay solution in the biosensor detector system under low CO₂ environment for at least 2 hrs.

20. The method of claim 6, wherein the concentration of the CO₂ environment in the biosensor detection system is below 3.5%.

21. The method of claim 6, wherein the cells are maintained and incubated with the assay solution with a carbonic anahydrase inhibitor in the biosensor detector system for 30 min, 1 hr, 2 hrs, 3 hrs, 4 hrs, 5 hrs, 6 hrs, 8 hrs, 10 hrs, 15 hrs, 20 hrs, 30 hrs, 40 hrs, 50 hrs, or 100 hrs.

22. The method of claim 21, wherein the cells are maintained and incubated with the assay solution with a carbonic anahydrase inhibitor in the biosensor detector system for at least 2 hrs.

23. A method of incubating cells on a biosensor, comprising the steps of:
a. providing a biosensor;
b. seeding cells onto the biosensor surface;
c. culturing cells under serum containing medium; and
d. contacting the cell with a test compound wherein the contact produces a biosensor response from the cell, and using the biosensor response to determine if the test compound is a PDE modulator.

24. The method of claim 6, wherein the cells are synchronized via culturing and wherein the cells are cultured for an extended period of time.

25. The method of claim 6, wherein the cells reach high confluency and quiescent state by being starved.


SEQUENCE LISTING (7 sequences)

SEQ ID NO: 1 - protein, 6 aa, Artificial Sequence
  Chemically synthesized
SLIGKV

SEQ ID NO: 2 - DNA, 20 bp, Artificial Sequence
  Chemically synthesized; primer for PDE3A
ctggccaaccttcaggaatc

SEQ ID NO: 3 - DNA, 21 bp, Artificial Sequence
  Chemically synthesized; primer of PDE3A
gcctcttggtttccctttctc

SEQ ID NO: 4 - DNA, 23 bp, Artificial Sequence
  Chemically synthesized; primer for PDE3B
aatcttggtctgcccatcagtcc

SEQ ID NO: 5 - DNA, 24 bp, Artificial Sequence
  Chemically synthesized; primer to PDE3B
ttcagtgaggtggtgcattagctg

SEQ ID NO: 6 - DNA, 27 bp, Artificial Sequence
  Chemically synthesized; primer for GAPDH
gaccccttcattgacctcaactacatg

SEQ ID NO: 7 - DNA, 24 bp, Artificial Sequence
  Chemically synthesized; primer for GAPDH
gtccaccaccctgttgctgtagcc